(19) United States
(12) Patent Application Publication
(10) Pub. No.: US 2017/0072173 A1
(43) Pub. Date: Mar. 16, 2017

(54) INFLATABLE TRANSLUMENAL SHUNTS AND METHODS AND DEVICES FOR DELIVERY

(71) Applicant: TREUS LEGACY PARTNERS, LLC, Redwood City, CA (US)

(72) Inventors: Jacques VAN DAM, Los Angeles, CA (US); Robert Matthew OHLINE, Redwood CIty, CA (US); James Craig MILROY, Palo Alto, CA (US)

(21) Appl. No.: 15/263,707

(22) Filed: Sep. 13, 2016

Related U.S. Application Data
(60) Provisional application No. 62/217,952, filed on Sep. 13, 2015.

(51) Int. Cl. A61M 27/00; A61M 25/01; A61M 25/06

(52) U.S. Cl. CPC A61M 27/002; A61M 25/065; A61M 25/01

(57) ABSTRACT

Devices and methods for treating biliary disease including an inflatable shunt configured for deployment between one of a lumen of a gallbladder or gallbladder duct and a second body lumen.

### CROSS-REFERENCE

This application claims the benefit of U.S. Provisional Application No. 62/217,952, filed Sep. 13, 2015, entitled INFLATABLE TRANSLUMENAL SHUNTS AND METHODS FOR DELIVERY which application is incorporated herein by reference.

### BACKGROUND

Field of the Invention

The disclosed solutions address challenges confronted in the use of shunts as in interconnect between two lumens within a body. More specifically, the shunt designs target the biliary system and target the treatment of biliary disease. Biliary disease includes conditions affecting the gallbladder, cystic duct, and common bile duct. More generally, the shunt designs are useful for interconnecting any two sufficiently proximal lumens within a body in a variety of medical fields.

Background

Several conditions within the body give rise to the need for treatment, drainage, and/or elimination of calculi within the biliary system, as well as other locations within the body of a patient. Treatments for biliary disease are the focus of the following discussion, though the devices and methods described herein are generally applicable in a wide variety of medical applications.

### Biliary System Function and Anatomy:

Bile is a greenish-brown digestive fluid produced by the liver 10 illustrated in FIG. 1, and is vital for the digestion of fatty foods. Bile is secreted by liver cells and collected by a network of ducts that converge at the common hepatic duct 12. While a small quantity of bile drains directly into the duodenum 30 (the first portion of the small intestine, immediately downstream of the stomach), the majority of the bile travels through the common hepatic duct 12 and accumulates in the gallbladder 14. Healthy gallbladders are pear-shaped sacs with a thin muscular wall that, on average, measure 10 cm in length and can store approximately 50 ml of fluid. When fatty foods are ingested, the hormone cholecystokinin is released, which causes the gallbladder 14 to contract. Contraction of the gallbladder 14 forces bile to flow from the gallbladder 14, through the cystic duct 16, into the common bile duct 18, out the papilla 28, and finally into the duodenum 30 of the small intestine. Here, it mixes and reacts with the chyme (partially digested food) that exits the stomach. The Sphincter of Oddi 26 controls secretions from the liver 10, pancreas 24, and gallbladder 14 into the duodenum 30 of the small intestine. The opening on the inside of the descending duodenum 30 after the Sphincter of Oddi 26 is called the major duodenal papilla 28 (of Vater). Together, the biliary ducts, the gallbladder 14, the cystic duct 16 and the common bile duct 18 comprise the biliary system (FIG. 1).

### Biliary Disease:

The most common problem that arises in the biliary system is the formation of gallstones, a condition called cholelithiasis. Approximately 20 million Americans have gallstones, and about 1-3% will exhibit symptoms in any given year. In the US, gallstones are more common among women, with 25% of women having gallstones by the age of 60 and 50% by the age of 75. Pregnancy and hormone replacement therapy increase the risk of forming gallstones. Prevalence is lower for American men: approximately 25% will develop gallstones by the age of 75. In the US, gallstones are responsible for the largest number of hospital admissions due to severe abdominal pain.

Gallstones 20, 20′ are most often composed of cholesterol, but may also be formed from calcium bilirubinate, in which case they are called pigment stones, as shown in FIG. 2. They range in size from a few millimeters to several centimeters, and are irregularly shaped solids resembling pebbles. They can form in the gallbladder 14, cystic duct 16, and/or the common bile duct 18. By themselves, gallstones 20 do not necessarily result in disease states. This is the case 90% of the time. However, stones can cause infection and inflammation, a condition known as cholecystitis, which is generally the result of restricting or blocking the flow of bile from the gallbladder 14 and common bile duct 18, or the fluids secreted by the pancreas 24 via the pancreatic duct 22.

Gallbladder disease may be chronic, and patients who suffer from this may periodically experience biliary colic. Symptoms include pain in the upper right abdomen near the ribcage, nausea, and/or vomiting. The pain may resolve within an hour of onset, may prove unresponsive to over-the-counter medicines, and may not decrease with changes of position or the passage of gas. Recurrence is common, with pain often recurring at the same time of day, but with frequency of less than once per week. Fatty or large meals may cause recurrence several hours after eating, often awakening the patient at night. Patients may elect to suffer from these symptoms for extended periods of time, such as years or even decades.

Patients with chronic cholecystitis have gallstones and low-grade inflammation. Untreated, the gallbladder 14 may become scarred and stiff over time, leading to a condition called dysfunctional gallbladder. Patients who have chronic cholecystitis or dysfunctional gallbladder may experience gas, nausea, and abdominal discomfort after meals, and chronic diarrhea.

In contrast to patients with chronic gallbladder disease, a small fraction (in the range of 1-3%) of patients symptomatic for gallstones develop acute cholecystitis (inflammation of the gallbladder) due to obstruction of the common bile duct 18 or cystic duct 16 by stones 20, 20′ or bile sludge. Symptoms are similar to biliary colic, though they are more severe and persistent. Pain in the upper right abdomen can be constant and severe, the intensity may increase when drawing breath, and it may last for days. Pain may radiate to the back, under the breastbone or the shoulder blades, and it may be perceived on the left side of the abdomen. In addition to nausea and vomiting, one third of patients experience fever and chills. Complications from acute cholecystitis can be serious and life-threatening, and include gangrene, abscesses, perforation of the gallbladder 14 which can lead to bile peritonitis, pus in the gallbladder wall (empyema), fistulae, and gallstone ileus (wherein a gallstone creates a blockage in the small intestine).

When gallstones 20′ become lodged in the common bile duct 18, the condition is known as choledocholithiasis. Symptoms for this condition include pain, nausea and vomiting, and some patients develop jaundice, have dark urine and/or lighter stools, rapid heartbeat, and experience an abrupt drop in blood pressure. These symptoms can also be accompanied by fever, chills, and/or severe pain in the upper right abdomen. Complications from choledocholithiasis can also be very serious, and include infection of the common bile duct 18 (cholangitis) and inflammation of the pancreas 24 (pancreatitis).

### Treatment of Biliary Disease:

The most effective treatment for biliary disease has been surgical removal of the gallbladder 14, a procedure called cholecystectomy. Surgical removal of the gallbladder 14 is indicated for patients who experience a number of less severe gallstone attacks, cholecystitis, choledocholithiasis, pancreatitis, acalculous biliary pain with evidence of impaired gallbladder 14 emptying, those at high risk for developing gallbladder cancer, and those who have previously undergone endoscopic sphincterotomy for common bile duct stones. Other treatment modalities exist and are frequently used, but gallbladder disease tends to recur in the majority of patients who forgo cholecystectomy and pursue alternatives. Removal of the gallbladder 14 is highly successful at permanently eliminating biliary disease. Cholecystectomy is one of the most commonly performed procedures on women. The gallbladder 14 is not an essential organ, and after a period of adjustment post-surgery, patients tend to return to more or less normal digestive function.

Cholecystectomy can be performed either as open surgery, which requires a single large incision in the upper right abdomen, or laparoscopic surgery, in which several small instruments are inserted through much smaller incisions in the abdomen. Over 90% of cholecystectomies are performed laparoscopically. The primary benefits of this minimally invasive approach are faster recovery for the patient and a reduction in overall healthcare costs. Patients who receive laparoscopic cholecystectomy are usually released from a hospital the same day as the procedure. By contrast, patients receiving open cholecystectomies typically spend 5-7 days in a hospital before release. 5-10% of laparoscopic procedures convert to open procedures when difficulties arise, such as injury to major blood vessels, inadequate access, inadequate visualization, previous endoscopic sphincterotomy, and thickened gallbladder wall. Complications from cholecystectomy (open or laparoscopic) include bile duct injuries (0.1-0.5% for open, 0.3-2% with a declining trend for laparoscopic), pain, fatigue, nausea, vomiting, and infection. In up to 6% of cases, surgeons fail to identify and remove all gallstones present.

In some cases, the degree of infection and inflammation prevents patients from undergoing immediate cholecystectomy. In these cases, the gallbladder 14 must be treated with antibiotics and anti-inflammatory agents, and drained through a tube into a reservoir outside the abdomen. Placement of this tube occurs in a procedure called percutaneous cholecystostomy, in which a needle is introduced to the gallbladder 14 through the abdomen, fluid is withdrawn, the needle puncture is dilated, and a drainage catheter is inserted. This catheter drains into an external bag which must be emptied several times a day until the tube is removed. The drainage catheter may be left in place for up to 8 weeks. In cases where no drainage catheter is inserted, the procedure is called gallbladder aspiration. Since no indwelling catheter is placed, the complication rate for gallbladder aspiration is lower than that of percutaneous cholecystostomy.

Treatment methodologies for gallbladder disease other than cholecystectomy include 1) expectant management, and 2) treatments that focus on the elimination of gallstones in the gallbladder, such as dissolution therapy and extracorporeal shockwave lithotripsy (ESWL). Lithotripsy is a general term referring to the act of breaking up stones. Tools used to break up stones are known as lithotripters. Lithotripsy may be performed using any of a variety of tools and methods, including mechanical lithotripsy (in which a mechanical tool is used to physically cut or break stones into smaller pieces), laser lithotripsy (in which laser energy is applied to stones to cause their destruction), electro-hydraulic lithotripsy or EHL (in which electrically produced shockwaves are applied directly to stones via a probe), and extracorporeal shockwave lithotripsy or ESWL (in which externally produced acoustic waves are focused on gallstones within the body).

When gallstones are present in the bile duct 18, rather than the gallbladder 14, other treatment methodologies are employed. The most common of these include: endoscopic retrograde cholangiopanctreatograpy (ERCP), with or without endoscopic sphincterotomy; mechanical lithotripsy; laser lithotripsy; and electro-hydraulic shockwave lithotripsy (EHL). For gallstones 20 located in the gallbladder 14, these treatments are generally infeasible, since the gallbladder is not accessible via endoscopy (e.g., endoscopic retrograde cholangiopancreatography).

ERCP (endoscopic retrograde cholangiopancreatograpy) is an endoscopic procedure for treating gallstones, obstructions, strictures, and other conditions in the common bile duct 18 and pancreatic duct 22. During ERCP, an endoscope is introduced through the mouth of a patient, past the stomach to the papilla 28, where the common bile duct 18 empties into the duodenum 30. The procedure most commonly involves inserting instruments and tools into the common bile duct 18 via the papilla 28 in order to treat biliary disease. Often, especially in cases where relatively large gallstones are removed via the bile duct, endoscopic sphincterotomy is performed, which is a procedure that enlarges the opening of the papilla 28 in the small intestine 30 surgically or via balloon dilation. Radiographic contrast agent is introduced into the common bile duct 18 to visualize the biliary tree fluoroscopically.

During ERCP, tools for clearing gallstones 20, 20′ and other blockages, such as endoscopic baskets, lithotripters, and balloon catheters, may be deployed to capture, break up, and extract gallstones 20, 20′ and/or the resulting debris, and remove bile sludge. Drainage catheters and stents may also be inserted to facilitate the drainage of bile past obstructions. ERCP is a technically challenging procedure, and complication rates of 5-8% have been reported. Complications include pancreatitis, infection, bleeding, and perforation.

### SUMMARY

An aspect of the disclosure is directed to inflatable shunts. Suitable inflatable shunts comprise: a first inflatable anchor at a first end having a first inflatable anchor interior surface and a first inflatable anchor exterior surface forming a first inflatable anchor lumen therebetween; a second inflatable anchor at a second end having a second inflatable anchor interior surface and a second inflatable anchor exterior surface forming a second inflatable anchor lumen therebetween; and an inflation port. Shunts can also comprise: a first inflatable anchor at a first end having a first inflatable anchor interior surface and a first inflatable anchor exterior surface forming a first inflatable anchor lumen therebetween; a second inflatable anchor at a second end having a second inflatable anchor interior surface and a second inflatable anchor exterior surface forming a second inflatable anchor lumen therebetween; a connecting section positioned between the first inflatable anchor and the second inflatable anchor; and an inflation port. The first inflatable anchor lumen and second inflatable anchor lumen can be contiguous such that only a single lumen is formed throughout the device. In some configurations, the inflation port is positionable on at least one of the first inflatable anchor exterior, the second inflatable anchor exterior, and the connecting section. One or more inflation ports can be provided which are positionable on a non-tissue contacting surface. The inflatable shunt can have a central lumen and/or be inflatable. Additionally the inflation port can have a central lumen. One or more activatable materials can be provided within at least one of the first inflatable anchor lumen, the second inflatable anchor lumen, and the connecting section. The inflatable shunt is inflated by one or more of a filling material and activation of an expandable material. At least one of the filling material and expandable material can be chosen based on a material characteristic which changes hardness of the material. The filling material can be one or more of each of a compressible material and an incompressible material. Moreover, the compressible material can be one or more of air, inert gases, carbon dioxide, foam and gel. The incompressible material can be one or more of liquid and gel. The exterior surface of the shunt can be configured to have one or more of each of a surface texture, a ridge, a rib, an aperture, and a barb. Additionally, a plug can be provided. The plug can be part of an inflation port or positioned within a central lumen. Additionally, the inflation port can be self-healing. In some configurations, at least a portion of the shunt is biodegradable. In other configurations, a radial expansion of the inflatable shunt is selectable. Additionally, a valve can be provided which is positionable within a lumen of the connecting section. A structural component can be provided or activated wherein the structural component either surrounds at least a portion of at least one of the first inflatable anchor, the second inflatable anchor, and the connecting section, or is incorporated into a portion of a wall of at least one of the first inflatable anchor, the second inflatable anchor and the connecting section. The first inflatable anchor and the second inflatable anchor can be configurable so that the anchors apply pressure to tissue positioned between a first inflatable anchor surface that is adjacent a second inflatable anchor surface. Thus the space between the surfaces of the anchors is less than the uncompressed thickness of the tissue.

Another aspect of the disclosure is directed to a collapsible inflatable shunt. Suitable collapsible inflatable shunts comprise: a first inflatable anchor at a first end having a first inflatable anchor interior surface and a first inflatable anchor exterior surface forming a first inflatable anchor lumen therebetween; a second inflatable anchor at a second end having a second inflatable anchor interior surface and a second inflatable anchor exterior surface forming a second inflatable anchor lumen therebetween, wherein the inflatable shunt has a delivery configuration and a deployed configuration. Additional collapsible inflatable shunts comprise: a first inflatable anchor at a first end having a first inflatable anchor interior surface and a first inflatable anchor exterior surface forming a first inflatable anchor lumen therebetween; a second inflatable anchor at a second end having a second inflatable anchor interior surface and a second inflatable anchor exterior surface forming a second inflatable anchor lumen therebetween; a connecting section positioned between the first inflatable anchor and the second inflatable anchor, wherein the inflatable shunt has a folded delivery configuration and an unfolded deployed configuration. Additionally, an inflation port can be provided. The inflation port can be positionable on at least one of the first inflatable anchor exterior, the second inflatable anchor exterior, and the connecting section. The first inflatable anchor lumen and second inflatable anchor lumen can be contiguous such that only a single lumen is formed throughout the device. In some configurations, the inflation port is positionable on at least one of the first inflatable anchor exterior, the second inflatable anchor exterior, and the connecting section. One or more inflation ports can be provided which are positionable on a non-tissue contacting surface. The inflatable shunt can have a central lumen and/or be inflatable. Additionally the inflation port can have a central lumen. One or more activatable materials can be provided within at least one of the first inflatable anchor lumen, the second inflatable anchor lumen, and the connecting section. The inflatable shunt is inflated by one or more of a filling material and activation of an expandable material. At least one of the filling material and expandable material can be chosen based on a material characteristic which changes hardness of the material. The filling material can be one or more of each of a compressible material and an incompressible material. Moreover, the compressible material can be one or more of air, inert gases, carbon dioxide, foam and gel. The incompressible material can be one or more of liquid and gel. The exterior surface of the shunt can be configured to have one or more of each of a surface texture, a ridge, a rib, an aperture, and a barb. Additionally, a plug can be provided. The plug can be part of an inflation port or positioned within a central lumen. Additionally, the inflation port can be self-healing. In some configurations, at least a portion of the shunt is biodegradable. In other configurations, a radial expansion of the inflatable shunt is selectable. Additionally, a valve can be provided which is positionable within a lumen of the connecting section. A structural component can be provided or activated wherein the structural component either surrounds at least a portion of at least one of the first inflatable anchor, the second inflatable anchor, and the connecting section, or is incorporated into a portion of a wall of at least one of the first inflatable anchor, the second inflatable anchor and the connecting section. The first inflatable anchor and the second inflatable anchor can be configurable so that the anchors apply pressure to tissue positioned between a first inflatable anchor surface that is adjacent a second inflatable anchor surface. Thus the space between the surfaces of the anchors is less than the uncompressed thickness of the tissue.

Yet another aspect of the disclosure is directed to an inflatable shunt comprising: a first inflatable anchor at a first end having a first inflatable anchor interior surface and a first inflatable anchor exterior surface forming a first inflatable anchor lumen therebetween; a second inflatable anchor at a second end having a second inflatable anchor interior surface and a second inflatable anchor exterior surface forming a second inflatable anchor lumen therebetween; and a structural component wherein the structural component either surrounds at least a portion of at least one of the first inflatable anchor, the second inflatable anchor and the connecting section, or is incorporated into a portion of a wall of at least one of the first inflatable anchor, the second inflatable anchor and the connecting section. Additional embodiments of inflatable shunt can comprise: a first inflatable anchor at a first end having a first inflatable anchor interior surface and a first inflatable anchor exterior surface forming a first inflatable anchor lumen therebetween; a second inflatable anchor at a second end having a second inflatable anchor interior surface and a second inflatable anchor exterior surface forming a second inflatable anchor lumen therebetween; a connecting section positioned between the first inflatable anchor and the second inflatable anchor; and a structural component wherein the structural component either surrounds at least a portion of at least one of the first inflatable anchor, the second inflatable anchor and the connecting section, or is incorporated into a portion of a wall of at least one of the first inflatable anchor, the second inflatable anchor and the connecting section. The first inflatable anchor lumen and second inflatable anchor lumen can be contiguous such that only a single lumen is formed throughout the device. In some configurations, the inflation port is positionable on at least one of the first inflatable anchor exterior, the second inflatable anchor exterior, and the connecting section. One or more inflation ports can be provided which are positionable on a non-tissue contacting surface. The inflatable shunt can have a central lumen and/or have an inflatable connecting section. Additionally the inflation port can have a central lumen. One or more activatable materials can be provided within at least one of the first inflatable anchor lumen, the second inflatable anchor lumen, and the connecting section. The inflatable shunt is inflated by one or more of a filling material and activation of an expandable material. At least one of the filling material and expandable material can be chosen based on a material characteristic which changes hardness of the material. The filling material can be one or more of each of a compressible material and an incompressible material. Moreover, the compressible material can be one or more of air, inert gases, carbon dioxide, foam and gel. The incompressible material can be one or more of liquid and gel. The exterior surface of the shunt can be configured to have one or more of each of a surface texture, a ridge, a rib, an aperture, and a barb. Additionally, a plug can be provided. The plug can be part of an inflation port or positioned within a central lumen. Additionally, the inflation port can be self-healing. In some configurations, at least a portion of the shunt is biodegradable. In other configurations, a radial expansion of the inflatable shunt is selectable. Additionally, a valve can be provided which is positionable within a lumen of at least one of the inflatable shunt and the inflation port. A structural component can be provided or activated wherein the structural component either surrounds at least a portion of at least one of the first inflatable anchor, the second inflatable anchor, and the connecting section, or is incorporated into a portion of a wall of at least one of the first inflatable anchor, the second inflatable anchor and the connecting section. The first inflatable anchor and the second inflatable anchor can be configurable so that the anchors apply pressure to tissue positioned between a first inflatable anchor surface that is adjacent a second inflatable anchor surface. Thus the space between the surfaces of the anchors is less than the uncompressed thickness of the tissue.

Still another aspect of the disclosure is directed to a method of treating biliary disease. Suitable methods comprise: creating a first otomy and a second otomy adjacent the first otomy; delivering an inflatable shunt to the first otomy and the second otomy wherein the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, and an inflation port; inflating the inflatable shunt; anchoring the first inflatable anchor on a first tissue surface; and anchoring the second inflatable anchor on a second tissue surface which does not contact the first tissue surface. Additional methods can comprise: creating a first otomy and a second otomy adjacent the first otomy; delivering an inflatable shunt to the first otomy and the second otomy wherein the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, a connecting section positioned between the first inflatable anchor and the second inflatable anchor, and an inflation port; inflating the inflatable shunt; anchoring the first inflatable anchor on a first tissue surface; and anchoring the second inflatable anchor on a second tissue surface which does not contact the first tissue surface. An otomy includes a cut into a part of the body by making an incision into or a cut through the body, such as a cut through the duodenum or a cut through the gallbladder which enables an inflatable shunt to be passed through the wall of the duodenum and the wall of the gallbladder. Additionally, the method can include delivering a filling material to the inflatable shunt. The filling material can be at least one of compressible and incompressible. Additionally a structural component can be delivered or activated. The shunt can also be delivered via an endoscope. Some methods include necrosing tissue in contact with the inflatable shunt by causing cell injury or cell death by applying pressure to the target tissue. Where the tissue is necrosed, the method can include removing the inflatable shunt and the necrosed tissue.

Still another method includes methods of treating biliary disease comprising: creating a first otomy and a second otomy adjacent the first otomy; delivering an inflatable shunt to the first otomy and the second otomy wherein the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, wherein the inflatable shunt has a delivery configuration and a deployed configuration; inflating the inflatable shunt; anchoring the first inflatable anchor on a first tissue surface; and anchoring the second inflatable anchor on a second tissue surface which does not contact the first tissue surface. Additional methods can comprise: creating a first otomy and a second otomy adjacent the first otomy; delivering an inflatable shunt to the first otomy and the second otomy wherein the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, a connecting section positioned between the first inflatable anchor and the second inflatable anchor, wherein the inflatable shunt has a folded delivery configuration and an unfolded deployed configuration; inflating the inflatable shunt; anchoring the first inflatable anchor on a first tissue surface; and anchoring the second inflatable anchor on a second tissue surface which does not contact the first tissue surface. Additionally, the method can include delivering a filling material to the inflatable shunt. The filling material can be at least one of compressible and incompressible. Additionally a structural component can be delivered or activated. The shunt can also be delivered via an endoscope. Some methods include necrosing tissue in contact with the inflatable shunt by causing cell injury or cell death by applying pressure to the target tissue. Where the tissue is necrosed, the method can include removing the inflatable shunt and the necrosed tissue.

Yet another method of treating biliary disease comprises: creating a first otomy and a second otomy adjacent the first otomy; delivering an inflatable shunt comprising a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, and a structural component wherein the structural component either surrounds at least a portion of at least one of the first inflatable anchor, the second inflatable anchor and the connecting section, or is incorporated into a portion of a wall of at least one of the first inflatable anchor, the second inflatable anchor and the connecting section; inflating the inflatable shunt; anchoring the first inflatable anchor on a first tissue surface; and anchoring the second inflatable anchor on a second tissue surface which does not contact the first tissue surface. Another method can include creating a first otomy and a second otomy adjacent the first otomy; delivering an inflatable shunt comprising a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, a connecting section positioned between the first inflatable anchor and the second inflatable anchor, and a structural component wherein the structural component either surrounds at least a portion of at least one of the first inflatable anchor, the second inflatable anchor and the connecting section, or is incorporated into a portion of a wall of at least one of the first inflatable anchor, the second inflatable anchor and the connecting section; inflating the inflatable shunt; anchoring the first inflatable anchor on a first tissue surface; and anchoring the second inflatable anchor on a second tissue surface which does not contact the first tissue surface. Additionally, the method can include delivering a filling material to the inflatable shunt. The filling material can be at least one of compressible and incompressible. Additionally a structural component can be delivered or activated. The shunt can also be delivered via an endoscope. Some methods include necrosing tissue in contact with the inflatable shunt by causing cell injury or cell death by applying pressure to the target tissue. Where the tissue is necrosed, the method can include removing the inflatable shunt and the necrosed tissue.

Another aspect of the disclosure is directed to a method for removing an inflatable shunt, comprising locating the inflatable shunt delivered to a first otomy and a second otomy adjacent the first otomy, and changing a profile of the inflatable shunt from a deployed profile at a removal profile wherein the deployed profiled has a larger circumference at a cross-section than the removal profile. The method can be achieved by, for example, deflating the inflatable shunt. Additionally, the method may include removing the inflatable shunt.

Still another aspect of the disclosure is directed to shunt inflation devices comprising: an inflation element wherein the inflation element engages an inflation port of an inflatable shunt at a distal end of the inflation element, wherein the inflatable shunt is at least one of the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, and an inflation port; the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, and a shunt, wherein the inflatable shunt has a delivery configuration and a deployed configuration; and the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, an inflation port and a structural component wherein the structural component either surrounds at least a portion of at least one of the first inflatable anchor, the second inflatable anchor and the connecting section, or is incorporated into a portion of a wall of at least one of the first inflatable anchor, the second inflatable anchor and the connecting section. The inflation element can be an elongated tubular member. Another configuration can include shunt inflation devices comprising: an inflation element wherein the inflation element engages an inflation port of an inflatable shunt at a distal end of the inflation element, wherein the inflatable shunt is at least one of the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, a connecting section positioned between the first inflatable anchor and the second inflatable anchor, and an inflation port; the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, a connecting section positioned between the first inflatable anchor and the second inflatable anchor, and a shunt, wherein the inflatable shunt has a folded delivery configuration and an unfolded deployed configuration; and the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, a connecting section positioned between the first inflatable anchor and the second inflatable anchor, an inflation port and a structural component wherein the structural component either surrounds at least a portion of at least one of the first inflatable anchor, the second inflatable anchor and the connecting section, or is incorporated into a portion of a wall of at least one of the first inflatable anchor, the second inflatable anchor and the connecting section. The inflation element can be an elongated tubular member. Additionally, the inflation element can be configured to be connectable to the inflatable shunt. In at least some configurations, the shunt inflation device further comprises a threaded distal end. The inflation element may also be removable from the inflation port by at least one of sliding, twisting, and severing. Additionally, the shunt inflation device can further comprise a one way flow control element and/or a removable distal end. The one way flow control element can be incorporated into an inflation port. In some configurations a stopper is provided. The shunt inflation device can further comprise a wire positioned within a central lumen of the inflation element and/or a collar positioned around at least a portion of an exterior wall of the inflation element. Additionally, the distal end of the shunt inflation device can have an angled distal end.

Another aspect of the disclosure is directed to a shunt inflation device comprising: an inflation element wherein the inflation element engages an inflation port of an inflatable shunt at a distal end of an elongated tubular member connectable to the inflatable shunt wherein the inflation element is removable from the inflation port of the inflation port by at least one of sliding, twisting and severing. The shunt inflation device can further comprise a threaded distal end and/or a one way flow control element and/or a removable distal end. The one way flow control element can be incorporated into an inflation port. In some configurations a stopper is provided. In other configurations, a wire positioned within a central lumen of the inflation element. The shunt inflation device can further comprise a collar positioned around at least a portion of an exterior wall of the inflation element. Additionally, the distal end of the shunt inflation device can be configured to have an angled distal end.

Yet another aspect of the disclosure is directed to a deliver system comprising: a needle; a dilation component; an inflatable shunt; an shunt delivery device. Additionally, the dilation component includes a cutting tool. In some configurations, the delivery system further comprises electrodes.

An aspect of the disclosure is directed to inflatable shunts. Suitable inflatable shunts comprise: a first inflatable anchor means at a first end having a first inflatable anchor means interior surface and a first inflatable anchor means exterior surface forming a first inflatable anchor means lumen therebetween; a second inflatable anchor means at a second end having a second inflatable anchor means interior surface and a second inflatable anchor means exterior surface forming a second inflatable anchor means lumen therebetween; and an inflation port. A connecting section means can be positioned between the first inflatable anchor means and the second inflatable anchor. The first inflatable anchor means lumen and second inflatable anchor means lumen can be contiguous such that only a single lumen is formed throughout the device. In some configurations, the inflation port means is positionable on at least one of the first inflatable anchor means exterior, the second inflatable anchor means exterior, and the connecting section means. One or more inflation port means can be provided which are positionable on a non-tissue contacting surface. The connecting section means can have a central lumen and/or be inflatable. Additionally the inflation port means can have a central lumen. One or more activatable materials can be provided within at least one of the first inflatable anchor means lumen, the second inflatable anchor means lumen, and the connecting section means. The inflatable shunt is inflated by one or more of a filling material and activation of an expandable material. At least one of the filling material and expandable material can be chosen based on a material characteristic which changes hardness of the material. The filling material can be one or more of each of a compressible material and an incompressible material. Moreover, the compressible material can be one or more of air, inert gases, carbon dioxide, foam and gel. The incompressible material can be one or more of liquid and gel. The exterior surface of the shunt can be configured to have one or more of each of a surface texture means, a ridge means, a rib means, an aperture, and a barb means. Additionally, a plug means can be provided. The plug means can be part of an inflation port means or positioned within a central lumen. Additionally, the inflation port means can be self-healing. In some configurations, at least a portion of the shunt is biodegradable. In other configurations, a radial expansion of the inflatable shunt is selectable. Additionally, a valve means can be provided which is positionable within a lumen of the connecting section means. A structural component can be provided or activated wherein the structural component either surrounds at least a portion of at least one of the first inflatable anchor, the second inflatable anchor, and the connecting section means, or is incorporated into a portion of a wall of at least one of the first inflatable anchor means, the second inflatable anchor means and the connecting section means. The first inflatable anchor means and the second inflatable anchor means can be configurable so that the anchor means apply pressure to tissue positioned between a first inflatable anchor means surface that is adjacent a second inflatable anchor means surface. Thus the space between the surfaces of the anchor means is less than the uncompressed thickness of the tissue.

Another aspect of the disclosure is directed to a collapsible inflatable shunt. Suitable collapsible inflatable shunts comprise: a first inflatable anchor means at a first end having a first inflatable anchor means interior surface and a first inflatable anchor means exterior surface forming a first inflatable anchor means lumen therebetween; a second inflatable anchor means at a second end having a second inflatable anchor means interior surface and a second inflatable anchor means exterior surface forming a second inflatable anchor means lumen therebetween; wherein the inflatable shunt has a delivery configuration and a deployed configuration. The inflatable shunt can also comprise a connecting section means positioned between the first inflatable anchor means and the second inflatable anchor means. Additionally, an inflation port means can be provided. The inflation port means can be positionable on at least one of the first inflatable anchor means exterior, the second inflatable anchor means exterior, and the connecting section. The first inflatable anchor means lumen and second inflatable anchor means lumen can be contiguous such that only a single lumen is formed throughout the device. In some configurations, the inflation port means is positionable on at least one of the first inflatable anchor means exterior, the second inflatable anchor means exterior, and the connecting section means. One or more inflation port means can be provided which are positionable on a non-tissue contacting surface. The connecting section means can have a central lumen and/or be inflatable. Additionally the inflation port means can have a central lumen. One or more activatable materials can be provided within at least one of the first inflatable anchor means lumen, the second inflatable anchor means lumen, and the connecting section. The inflatable shunt is inflated by one or more of a filling material and activation of an expandable material. At least one of the filling material and expandable material can be chosen based on a material characteristic which changes hardness of the material. The filling material can be one or more of each of a compressible material and an incompressible material. Moreover, the compressible material can be one or more of air, inert gases, carbon dioxide, foam and gel. The incompressible material can be one or more of liquid and gel. The exterior surface of the shunt can be configured to have one or more of each of a surface texture means, a ridge means, a rib means, an aperture, and a barb means. Additionally, a plug means can be provided. The plug means can be part of an inflation port means or positioned within a central lumen. Additionally, the inflation port means can be self-healing. In some configurations, at least a portion of the shunt is biodegradable. In other configurations, a radial expansion of the inflatable shunt is selectable. Additionally, a valve means can be provided which is positionable within a lumen of the connecting section. A structural component can be provided or activated wherein the structural component either surrounds at least a portion of at least one of the first inflatable anchor, the second inflatable anchor, and the connecting section, or is incorporated into a portion of a wall of at least one of the first inflatable anchor, the second inflatable anchor means and the connecting section. The first inflatable anchor means and the second inflatable anchor means can be configurable so that the anchors apply pressure to tissue positioned between a first inflatable anchor means surface that is adjacent a second inflatable anchor means surface. Thus the space between the surfaces of the anchors is less than the uncompressed thickness of the tissue.

Yet another aspect of the disclosure is directed to an inflatable shunt comprising: a first inflatable anchor means at a first end having a first inflatable anchor means interior surface and a first inflatable anchor means exterior surface forming a first inflatable anchor means lumen therebetween; a second inflatable anchor means at a second end having a second inflatable anchor means interior surface and a second inflatable anchor means exterior surface forming a second inflatable anchor means lumen therebetween; and a structural component wherein the structural component either surrounds at least a portion of at least one of the first inflatable anchor, the second inflatable anchor means and the connecting section, or is incorporated into a portion of a wall of at least one of the first inflatable anchor, the second inflatable anchor means and the connecting section. Additionally, the shunt can comprise a connecting section means positioned between the first inflatable anchor means and the second inflatable anchor means, The first inflatable anchor means lumen and second inflatable anchor means lumen can be contiguous such that only a single lumen is formed throughout the device. In some configurations, the inflation port means is positionable on at least one of the first inflatable anchor means exterior, the second inflatable anchor means exterior, and the connecting section means. One or more inflation port means can be provided which are positionable on a non-tissue contacting surface. The connecting section means can have a central lumen and/or be inflatable. Additionally the inflation port means can have a central lumen. One or more activatable materials can be provided within at least one of the first inflatable anchor means lumen, the second inflatable anchor means lumen, and the connecting section. The inflatable shunt is inflated by one or more of a filling material and activation of an expandable material. At least one of the filling material and expandable material can be chosen based on a material characteristic which changes hardness of the material. The filling material can be one or more of each of a compressible material and an incompressible material. Moreover, the compressible material can be one or more of air, inert gases, carbon dioxide, foam and gel. The incompressible material can be one or more of liquid and gel. The exterior surface of the shunt can be configured to have one or more of each of a surface texture means, a ridge means, a rib means, an aperture, and a barb means. Additionally, a plug means can be provided. The plug means can be part of an inflation port means or positioned within a central lumen. Additionally, the inflation port means can be self-healing. In some configurations, at least a portion of the shunt is biodegradable. In other configurations, a radial expansion of the inflatable shunt is selectable. Additionally, a valve means can be provided which is positionable within a lumen of the connecting section. A structural component can be provided or activated wherein the structural component either surrounds at least a portion of at least one of the first inflatable anchor, the second inflatable anchor, and the connecting section, or is incorporated into a portion of a wall of at least one of the first inflatable anchor, the second inflatable anchor means and the connecting section. The first inflatable anchor means and the second inflatable anchor means can be configurable so that the anchors apply pressure to tissue positioned between a first inflatable anchor means surface that is adjacent a second inflatable anchor means surface. Thus the space between the surfaces of the anchors is less than the uncompressed thickness of the tissue.

Still another aspect of the disclosure is directed to a method of treating biliary disease. Suitable methods comprise: creating a first otomy and a second otomy adjacent the first otomy; delivering an inflatable shunt to the first otomy and the second otomy wherein the inflatable shunt comprises a first inflatable anchor means at a first end having a first inflatable anchor means interior surface and a first inflatable anchor means exterior surface forming a first inflatable anchor means lumen therebetween, a second inflatable anchor means at a second end having a second inflatable anchor means interior surface and a second inflatable anchor means exterior surface forming a second inflatable anchor means lumen therebetween, and an inflation port; inflating the inflatable shunt; anchoring the first inflatable anchor means on a first tissue surface; and anchoring the second inflatable anchor means on a second tissue surface which does not contact the first tissue surface. The inflatable shunt can further comprise a connecting section means positioned between the first inflatable anchor means and the second inflatable anchor. An otomy includes a cut into a part of the body by making an incision into or a cut through the body, such as a cut through the duodenum or a cut through the pancreas which enables an inflatable shunt to be passed through the wall of the duodenum and the wall of the pancreas. Additionally, the method can include delivering a filling material to the inflatable shunt. The filling material can be at least one of compressible and incompressible. Additionally a structural component can be delivered or activated. The shunt can also be delivered via an endoscope. Some methods include necrosing tissue in contact with the inflatable shunt by causing cell injury or cell death by applying pressure to the target tissue. Where the tissue is necrosed, the method can include removing the inflatable shunt and the necrosed tissue.

Still another methods includes methods of treating biliary disease comprising: creating a first otomy and a second otomy adjacent the first otomy; delivering an inflatable shunt to the first otomy and the second otomy wherein the inflatable shunt comprises a first inflatable anchor means at a first end having a first inflatable anchor means interior surface and a first inflatable anchor means exterior surface forming a first inflatable anchor means lumen therebetween, a second inflatable anchor means at a second end having a second inflatable anchor means interior surface and a second inflatable anchor means exterior surface forming a second inflatable anchor means lumen therebetween, wherein the inflatable shunt has a delivery configuration and a deployed configuration; inflating the inflatable shunt; anchoring the first inflatable anchor means on a first tissue surface; and anchoring the second inflatable anchor means on a second tissue surface which does not contact the first tissue surface. Additionally, the method can include delivering a filling material to the inflatable shunt. The filling material can be at least one of compressible and incompressible. Additionally a structural component can be delivered or activated. The shunt can also be delivered via an endoscope. Some methods include necrosing tissue in contact with the inflatable shunt by causing cell injury or cell death by applying pressure to the target tissue. Where the tissue is necrosed, the method can include removing the inflatable shunt and the necrosed tissue.

Yet another method of treating biliary disease comprises: creating a first otomy and a second otomy adjacent the first otomy; delivering an inflatable shunt comprising a first inflatable anchor means at a first end having a first inflatable anchor means interior surface and a first inflatable anchor means exterior surface forming a first inflatable anchor means lumen therebetween, a second inflatable anchor means at a second end having a second inflatable anchor means interior surface and a second inflatable anchor means exterior surface forming a second inflatable anchor means lumen therebetween, and a structural component wherein the structural component either surrounds at least a portion of at least one of the first inflatable anchor, the second inflatable anchor means and the connecting section, or is incorporated into a portion of a wall of at least one of the first inflatable anchor, the second inflatable anchor means and the connecting section means; inflating the inflatable shunt; anchoring the first inflatable anchor means on a first tissue surface; and anchoring the second inflatable anchor means on a second tissue surface which does not contact the first tissue surface. The inflatable shunt can also include a connecting means. Additionally, the method can include delivering a filling material to the inflatable shunt. The filling material can be at least one of compressible and incompressible. Additionally a structural component can be delivered or activated. The shunt can also be delivered via an endoscope. Some methods include necrosing tissue in contact with the inflatable shunt by causing cell injury or cell death by applying pressure to the target tissue. Where the tissue is necrosed, the method can include removing the inflatable shunt and the necrosed tissue.

Still another aspect of the disclosure is directed to shunt inflation devices comprising: an inflation element wherein the inflation element engages an inflation port means of an inflatable shunt at a distal end of the inflation element, wherein the inflatable shunt is at least one of the inflatable shunt comprises a first inflatable anchor means at a first end having a first inflatable anchor means interior surface and a first inflatable anchor means exterior surface forming a first inflatable anchor means lumen therebetween, a second inflatable anchor means at a second end having a second inflatable anchor means interior surface and a second inflatable anchor means exterior surface forming a second inflatable anchor means lumen therebetween, and an inflation port; the inflatable shunt comprises a first inflatable anchor means at a first end having a first inflatable anchor means interior surface and a first inflatable anchor means exterior surface forming a first inflatable anchor means lumen therebetween, a second inflatable anchor means at a second end having a second inflatable anchor means interior surface and a second inflatable anchor means exterior surface forming a second inflatable anchor means lumen therebetween, and a shunt, wherein the inflatable shunt has a delivery configuration and a deployed configuration; and the inflatable shunt comprises a first inflatable anchor means at a first end having a first inflatable anchor means interior surface and a first inflatable anchor means exterior surface forming a first inflatable anchor means lumen therebetween, a second inflatable anchor means at a second end having a second inflatable anchor means interior surface and a second inflatable anchor means exterior surface forming a second inflatable anchor means lumen therebetween, an inflation port means and a structural component wherein the structural component either surrounds at least a portion of at least one of the first inflatable anchor, the second inflatable anchor means and the connecting section, or is incorporated into a portion of a wall of at least one of the first inflatable anchor, the second inflatable anchor means and the connecting section. The inflatable shunt can further include a connecting means between the first inflatable anchor means and the second inflatable anchor means. The inflation element can be an elongated tubular member. Additionally, the inflation element can be configured to be connectable to the inflatable shunt. In at least some configurations, the shunt inflation device further comprises a threaded distal end. The inflation element may also be removable from the inflation port means by at least one of sliding, twisting, and severing. Additionally, the shunt inflation device can further comprise a one way flow control element and/or a removable distal end. The one way flow control element can be incorporated into an inflation port. In some configurations a stopper is provided. The shunt inflation device can further comprise a wire positioned within a central lumen of the inflation element and/or a collar positioned around at least a portion of an exterior wall of the inflation element. Additionally, the distal end of the shunt inflation device can have an angled distal end.

Another aspect of the disclosure is directed to a shunt inflation device comprising: an inflation element wherein the inflation element engages an inflation port means of an inflatable shunt at a distal end of an elongated tubular member connectable to the inflatable shunt wherein the inflation element is removable from the inflation port means of the inflation port means by at least one of sliding, twisting and severing. The shunt inflation device can further comprise a threaded means at a distal end and/or a one way flow control means and/or a removable distal end. In some configurations a stopper means is provided. In other configurations, a wire means positioned within a central lumen of the inflation element. The shunt inflation device can further comprise a collar means positioned around at least a portion of an exterior wall of the inflation element. Additionally, the distal end of the shunt inflation device can be configured to have an angled distal end.

Yet another aspect of the disclosure is directed to a deliver system comprising: a needle means; a dilation means; an inflatable means; an shunt delivery means. Additionally, the dilation means includes a cutting means. In some configurations, the delivery system further comprises electrode means.

### INCORPORATION BY REFERENCE

All publications, patents, and patent applications mentioned in this specification are herein incorporated by reference to the same extent as if each individual publication, patent, or patent application was specifically and individually indicated to be incorporated by reference. See, for example, U.S. Pat. No. 9,282,968 issued Mar. 15, 2016, to Van Dam et al. for Applicator for Endoscopic Treatment of Biliary Disease; US2006/01677482 A1 published Jul. 27, 2006, by Swain et al. for Device for Transfixing and Joining Tissue; US 2009/0143713 A1 published Jun. 4, 2009 by Van Dam et al., for Biliary Shunts, Delivery Systems, Methods of Using the Same and Kits therefor; US2012/013041 A1 published May 24, 2012, by Lepulu et al. for Apparatus and Method for Penetrating and Enlarging Adjacent Tissue Layers; US2014/0018722 A1 published Jan. 16, 2014 by Scott et al. for Apparatus and Method for Creating a Lumen of a Desired Shape and Size in a Hollow Viscous Organ from Tissue of the Organ; WO 2012/007044 A1 published Jan. 19, 2012 by Pastorelli et al. for A Device for Translumenal Diversion of Bile; and WO 2012/007047 A1 published Jan. 19, 2012, by Stokes et al. for A Device and Method for Directing Bile from the Gallbladder in the Intestine.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the biliary system;

FIG. 2 illustrates gallstones in gallbladder, cystic duct, common bile duct;

FIGS. 3A-D illustrate an embodiment of an inflatable shunt;

FIG. 4 illustrates an embodiment of an inflatable shunt with a radially expanding center connecting section;

FIG. 5A illustrates internal structural connections to provide a shape to the inflatable shunt from a side view with seams or spot welds; FIG. 5B illustrates internal structural connections provide a shape to the inflatable shunt from a top view; FIG. 5C illustrates internal structural connections to provide a shape to the inflatable shunt from a top view; FIG. 5D illustrates internal structural connections to shape balloon: curved seam welds, top view; FIG. 5E illustrates internal structural connections to shape balloon: spiral seam welds on tube, top view; FIG. 5F illustrates internal structural connections to shape balloon: spiral seam welds on flats, top view;

FIG. 6 illustrates an inflation element of an embodiment (a tube that delivers a fluid);

FIGS. 7A-D illustrate detachment of inflation element from inflation port;

FIGS. 8A-B illustrate closing and sealing the inflation element with a stopper;

FIG. 9 illustrates kinking and clamping of the inflation element;

FIGS. 10A-B illustrate adhesive sealing of the inflation port;

FIG. 11 illustrates a self-healing inflation patch region;

FIGS. 12A-D illustrate toroidal donut-shaped anchors;

FIG. 13 illustrates toroidal anchors of differing sizes;

FIGS. 14A-B illustrate spherical anchors;

FIGS. 15A-B illustrate an embodiment with round distal anchor and toroidal proximal anchor;

FIGS. 16A-B illustrate an embodiment with toroidal distal anchor and round proximal anchor;

FIGS. 17A-B illustrate a balloon shunt constrained by outer structure;

FIG. 18 illustrates a balloon shunt filling structural component, resulting in tissue clamping forces;

FIGS. 19A-D illustrate packing the shunt to minimize profile for delivery;

FIG. 20 illustrates a delivery method flow chart;

FIG. 21 illustrates a delivery system;

FIG. 22 illustrates an optional sheath over inflatable shunt on delivery system

FIGS. 23A-C illustrate inflatable shunt inflation chamber configurations, with three separate inflation lumens and ports, and with a single inflation lumen and port, and a single inflation lumen and port, with lumens interconnected by small tubes;

FIG. 24 illustrates a dilation of central connecting section;

FIG. 25 illustrates a second dilation of tract through central connecting section of shunt;

FIG. 26 illustrates a method of delivery system removal; and

FIGS. 27A-C, 27D(1), 27D(2), and 27E illustrate valve configurations for controlling flow between connected lumens.

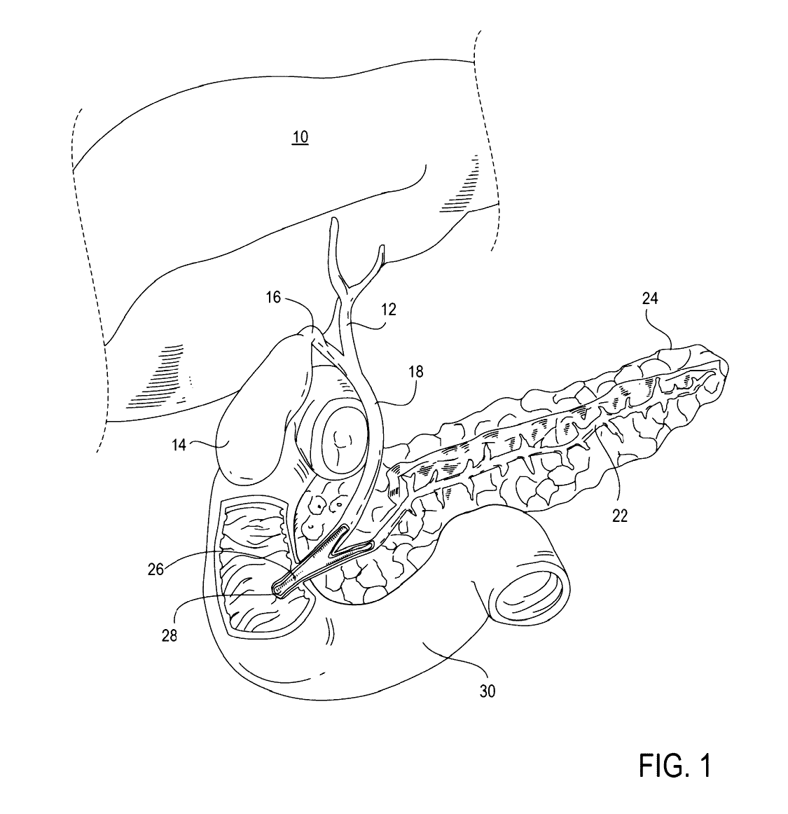

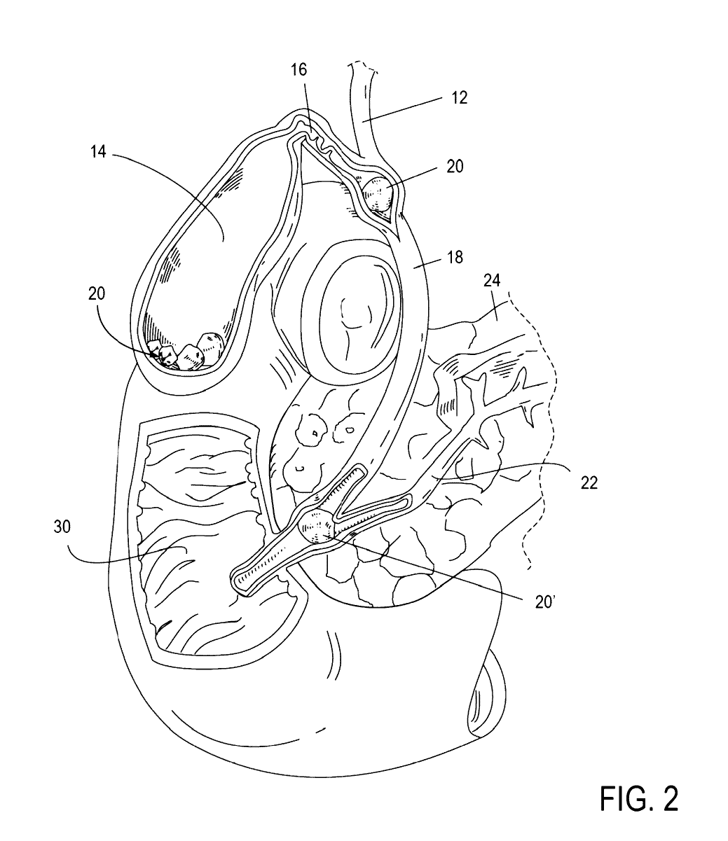

### DETAILED DESCRIPTION

The disclosed shunts are anastomotic devices that join two physiological lumens. The disclosed shunts can obviate the need for a variety of procedures, including: (1) percutaneous cholecystostomy, (2) cholecystectomy, (3) percutaneous trans-hepatic cholangiography (PTHC), and (4) endoscopic retrograde cholangiopancreatography (ERCP). Additionally, new treatment modalities are enabled for distal common bile duct obstruction secondary to pancreatic carcinoma, cholagiocarcinoma, and/or ampullary carcinoma. A conventional standard of care for treating biliary disease has been surgical removal of the gallbladder and closure of the cystic duct. While this has proven to be an effective way of permanently eliminating biliary disease and its recurrence, the present disclosed devices and methods accomplish the same end in a less invasive and less costly way. This may be achieved by treating biliary disease without requiring the removal of the gallbladder. Methods and apparatus are described in this application that are intended to effectively treat biliary disease with the gallbladder and cystic duct left in situ.

Treating biliary disease can employ using an endoscope to access a region in the gastrointestinal (GI) tract to which the gallbladder lumen is in close proximity (preferably the duodenum or stomach), locating the gallbladder, accessing the gallbladder through the wall, and then treating the underlying condition that led to the need for intervention. Treatments may include, but are not limited to, the following: providing drainage of the gallbladder and/or the biliary tree, delivering antibiotics and/or anti-inflammatory agents (short-term acting, fast acting, or time release) to the gallbladder and/or biliary tree, removing gallstones, facilitating the destruction and subsequent removal of gallstones, clearing obstructions, delivering catheters, delivering stents, temporarily or permanently defunctionalizing the gallbladder, temporarily or permanently defunctionalizing the cystic duct, and/or providing an alternate route for bile and/or other materials and fluids to be delivered to the intestine. Devices and therapies are preferably delivered in a single treatment, with minimal likelihood of or necessity for follow-up or repeat procedures.

Localization of the gallbladder can be performed via endoscopic ultrasound (EUS) through the wall of the GI tract, but may also be achieved by any other method that visualizes anatomical features, such as fluoroscopy, x-rays, magnetic resonance imaging (MM), computed axial tomography (CT) scans, ultrasound imaging from outside the body, or any other suitable method of anatomical imaging and visualization.

Once the gallbladder has been located, it may be accessed and/or treated through the wall of the GI tract (or any lumen in proximity to the gallbladder) by using tools and devices delivered preferably by means of the endoscope. Such tools and devices may be inserted down the length of the endoscope's working channel, or loaded onto or near the distal end of the endoscope. Alternately, tools and other devices may be used that do not require the aid of the endoscope for navigation or delivery. Direct visualization may be provided by the endoscope during the procedure, as well as irrigation, suction, and insufflation.

Though a location for accessing the gallbladder lumen is the duodenum, it may also be readily achieved through the wall of other regions of the GI tract, such as the stomach or the jejunum. Any lumen in close proximity to the gallbladder is a candidate for access to and treatment of the gallbladder and other members of the biliary system.

### Description of the Devices:

The present devices and methods enable in situ treatment of the gallbladder via the creation of a passageway between the gallbladder lumen and a lumen in close proximity, preferably the duodenum or stomach. This passageway may be created by use of device such as an inflatable implantable shunt device. Such a device would also enable connection of any two lumens in close proximity, such as a pancreatic pseudocyst and stomach, stomach and jejunum, and the like.

The passageway created by the device may be temporary or permanent. It may be thought of as a fistula that is intentionally created between a first lumen (e.g., the gallbladder) and a second lumen in proximity (e.g., the duodenum). Alternately, it may be thought of as a stoma between the first lumen (e.g., gallbladder) and second lumen (e.g., duodenum). The passageway created serves as a conduit, and also optionally as an access port through which a number of actions may be accomplished, drainage may be achieved, and treatments may be delivered.

A device forming the passageway may be left in the patient for a short period of time, such as a few days or weeks, or it may be left in place for extended periods of time, such as several weeks, months, or years. The device may also be left in place permanently. Tissue may form around the device, creating a fistula (e.g., passage or duct) that joins the connected lumens which may persist even if the connecting device is removed. The fistula may be beneficial and useful, as it may continue to drain the contents of one lumen (e.g., the gallbladder) into another lumen (e.g., the duodenum). It may provide either the primary or a secondary mechanism of delivering bile into the digestive system, for example. It may also provide convenient access in cases where repeated treatments are required. Though there may be no need to close the resulting fistula, it may also be closed at any time by a clinician should this become desirable. After removal of the shunt device that initially created the passageway, a fistula may remain open for some period of time and then close on its own, and may pose no additional risk and prove to be an acceptable course of events. Whether the device is left in place or removed, and whether the fistula is left open or closed, evidence at the site may serve to mark the location of treatment in the event of future procedures.

To facilitate delivery and deployment of a device, it may be useful to reconfigure its shape. For example, the cross-sectional area presented by the device at various locations (its “profile”) may be reduced. Inflatable shunt designs are especially suitable for this, since their profile may be quite small when deflated and carefully “packed”. In cases where the configuration of the device is caused to change, it may be helpful to conceive of the device having at least two configurations, for example: one configuration when it is delivered (a “delivery” or “initial” configuration), and another configuration when it is deployed in place and functional (a “deployed” or “final” configuration). Still other configurations may also be necessary or useful. For the delivery configuration, it may be advantageous to alter the cross-sectional area or profile (e.g. reduce the profile by deflating, folding, pleating, or otherwise packing the device), so that it more easily fits delivery mechanisms, such as the working channel of an endoscope. During deployment, the configuration of the device may be altered so that placement into the patient is facilitated and the intended function of the device achieved by the device in its final or deployed configuration.

### Description of the Inflatable Transluminal Shunt:

A transluminal shunt that is inflatable is disclosed, e.g., a balloon, or an arrangement of one or more balloons. The inflatable portion(s) of the device may be inflated by introducing a filling material, or by activating an expandable material that is either introduced during the procedure or is resident within the device prior to the procedure. In cases where material is introduced during a procedure, this may be done through a single inflation port, or separately through multiple ports.

The inflatable elements of the shunt may be inflated by introducing compressible materials (e.g., air, inert gases, carbon dioxide, foams, gels), or incompressible or nearly incompressible materials (e.g., water, saline, other liquids, gels). Alternately, the inflatable elements may be filled with an activatable substance that initially occupies a small volume during device delivery, and which may be selectively activated to occupy a larger volume during or after delivery. Materials may also be selected so that they change hardness or stiffness or state of matter (liquid-to-solid, for example), rather than or in addition to changes in volume that inflate the balloon elements. In this way, the shunt may be delivered in a soft configuration, and then changed into a stiffer configuration during delivery. Material used to alter the volume or stiffness of the inflatable elements may be biocompatible, so that exposing body tissue or fluids to the materials will not cause injury, toxicity, or other harm. However, biocompatibility is not a strict requirement, as long as the material is kept away from body tissue. Materials may be further selected so that they are radio-opaque and may be clearly visualized with fluoroscopy or other X-ray imaging technique, and/or are echogenic, so that they may be visualized with ultrasound.

The material used to fabricate the inflatable shunt may be elastic (e.g., elastomeric, latex, silicone, polyurethane) or inelastic (e.g., polyethylene terephthalate (Mylar®, from Dupont), nylon, polyethylene). The material is selected so that the uninflated and packed configuration allows the device to be made to be compact enough to facilitate delivery (e.g., through the tool channel of an endoscope) in its initial or delivery configuration, and the inflated (e.g., deployed or final) configuration may be made to assume the desired shape, and, in some cases, become rigid, and in still other cases, apply a desired degree of pressure to the lumen walls approximated by the anchors.

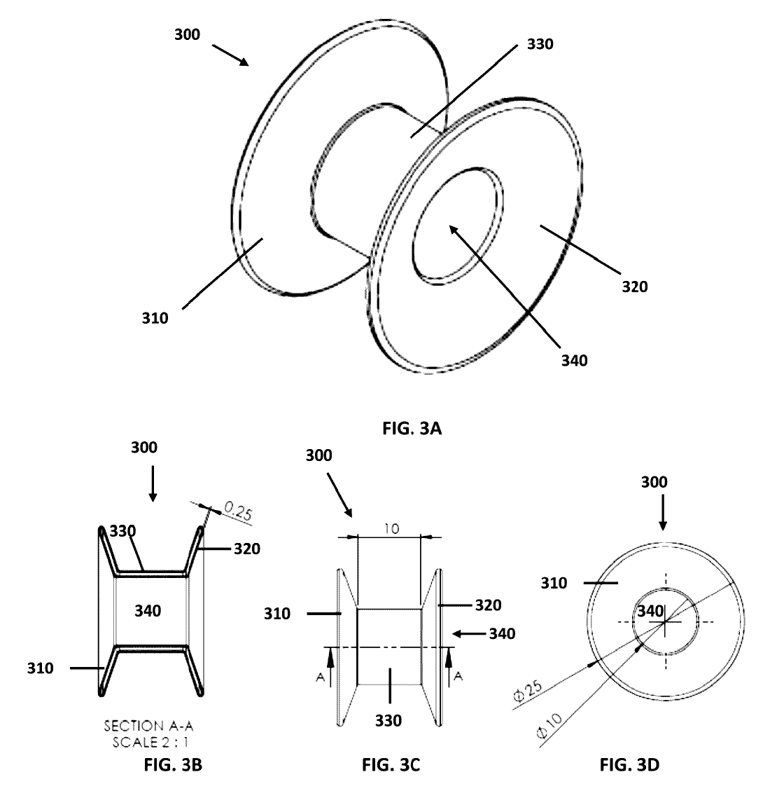

Many configurations of the proposed inflatable transluminal shunt are possible, and several representative arrangements are enumerated herein. An embodiment of the proposed inflatable shunt device 300 shown in FIGS. 3A-3D is comprised of inflatable anchors 310, 320 located at either end of a central connecting section 330. The central connecting section 330 may be configured in a number of ways, such as having length sufficient to span the thickness of the tissue walls to be joined (e.g., 10 mm, as shown in FIG. 3C), and optionally an inner lumen 340 with diameter sufficient for desired material(s) to pass through (e.g., 10 mm, as shown in FIG. 3D), and also optionally to provide access between lumens for endoscopes or tools. The outer diameter of the inflatable anchors 310, 320 is approximately 25 mm, with an approximate inner diameter of 10 mm as shown in FIG. 3D. The dimensions given in the figures and this description are representative, and only serve to indicate a typical size. It will be understood that these dimensions may vary slightly or substantially, without deviating from the disclosure.

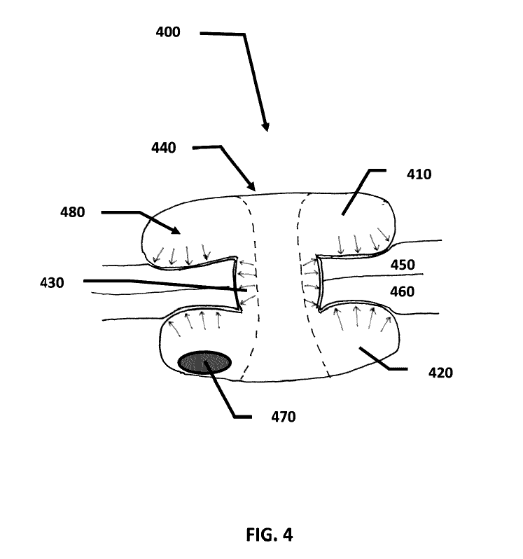

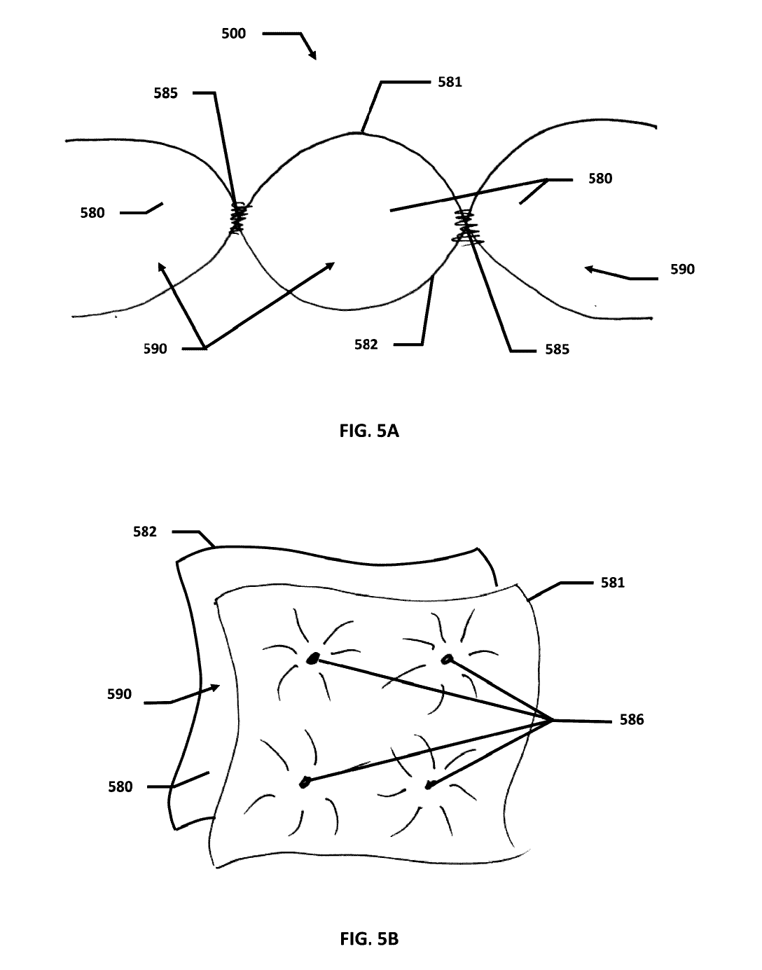

The inflatable anchors 310, 320, once inflated, hold the device securely in position across the lumen walls, and may be configured to apply pressure to at least a portion of the adjacent tissue so that the anchors more firmly approximate the walls and better seal the newly created junction shown in FIG. 4. In the configuration of FIG. 4, the pressure applied to adjacent tissue 450, 460 contacted by the anchors 410, 420 which is sufficient to prevent blood circulation in the adjacent tissue 450, 460, inducing pressure necrosis in the approximated tissue. Once necrosis occurs, the necrosed tissue detaches from the surrounding viable tissue, and the necrosed region, including the inflatable shunt 400, separates entirely from the lumen walls. The detached tissue and inflatable shunt 400 may then pass out of the body (e.g., by peristalsis in the intestine), obviating the need to perform additional procedures to manipulate, maintain, or remove the shunt.

The anchors 410, 420 may optionally be independently inflatable, so that, for example, when the distal end of the device is positioned as desired within the mammalian body, one anchor 410 may be inflated first, the device position may then be adjusted to ensure that it is in the desired location within the body, and subsequently the second anchor 420 may be inflated, securing the device in position across the walls of the joined lumens of the adjacent tissue 450, 460. Alternatively, both anchors 410, 420 may be inflated at the same time. For example, they may share a common interior volume that, when filled, inflates both anchors together.

The body lumens joined by the shunt (e.g., the gallbladder and the duodenum) may be used to guide the selection of the length of the central connecting section 430. For example, when connecting the gallbladder to the duodenum, a short (e.g., 2-4 mm) central connecting section 430 may be desired, whereas a central connecting section 430 which is longer (e.g. 5-10 mm) may be desired for connecting the stomach to the gallbladder.

In some cases, it may be desirable for the surfaces of the inflatable shunt 400 that is contacting the adjacent tissue 450, 460 to be smooth, have gentle radii, and not incorporate any sharp features, to avoid causing trauma to the tissue. For example, any seams and inflation ports could be located in areas on the device where they do not make contact with tissue (e.g., outer (non-tissue contacting) surfaces abutting the anchors 410, 420, or the inner lumen 440 of the central connecting section 430). Similarly, rough surfaces and/or sharp corners, if any are included in the device, may be located in these regions (or, more generally, apart from tissue-contacting elements of the device), away from tissue whenever possible, or to the maximum extent possible.

In other cases, it may be desirable for the surfaces of the inflatable shunt 400 that contacts the adjacent tissue 450, 460 to incorporate features that accommodate, encourage, or stimulate tissue ingrowth and/or attachment. For metallic stent technology, uncovered metallic stents allow for ingrowth of tissue, and are thus less likely to migrate over time. For the present inflatable shunt 400, features may be incorporated into the material comprising the inflatable shunt 400, or at least a part of the surface of the device, that produce an analogous result. Such surface features may include textures, ridges, ribs, holes, barbs, and the like, any or all of which would serve to engage the adjacent tissue and prevent migration of the inflatable shunt 400 from the implanted location. Similarly, substances may be inserted into the material comprising the outer layer of the inflatable shunt 400, or coatings applied to the surface of the inflatable shunt 400, that encourage or stimulate tissue growth and/or scar tissue formation, analogous to the construction and action of a drug eluting stent in cardiac applications.

Over time, scar tissue surrounds the junction in the tissue (e.g., as shown in FIG. 4, the region where the wall tissue of a first lumen 450 and the wall tissue of a second lumen 460 contact the central connecting section 430) and a fistula forms, after which the risk of leakage at the site is minimized. Removal of the shunt may be achieved by piercing or otherwise deflating the inflatable elements (e.g., first anchor 410, second anchor 420, and central connecting section 430) so that the cross-sectional area of the inflatable shunt 400 and/or its stiffness is reduced, after which the device may be withdrawn from the body. Further, the inflatable shunt 400, or at least some portion of the inflatable shunt 400 (e.g., a separate region, such as a “plug” 470) may optionally be comprised of a biodegradable or resorbable material, so that after a desired residence time within a body, the balloon will deflate and be expelled from the body without requiring additional intervention. It should be appreciated that such a region or construction may be incorporated into any of the designs described herein, including similar devices not specifically included, described, or enumerated.

Minimizing the volume, stiffness, and/or cross-sectional profile of the inflatable elements or anchors 410, 420, and central connecting section 430 of the inflatable shunt 400 will facilitate removal or repositioning of the shunts when desired. When inflation media or materials 480 such as compressible materials (which may or may not be biocompatible, e.g., gases, foams, gels), and/or incompressible or nearly incompressible materials (e.g., water, saline, gels), are used to inflate the anchors 410, 420, and central connecting section 430 of the inflatable shunt 400, the inflatable elements may be pierced, ruptured, or otherwise compromised to release the enclosed inflation material(s) in order to reduce or minimize their size for withdrawal. When the materials 480 used to fill or stiffen the inflatable elements are non-biocompatible materials, the materials may be withdrawn through a syringe, tube, catheter, or the like, without allowing the material(s) 480 to come in contact with patient tissue. When activatable material(s) 480 are selected, the material(s) and activation process may be selected such that the process is reversible, or multi-step, so that, for example, one step may activate the activatable material 480 and inflate the inflatable shunt 400, and a subsequent step may deactivate the activatable material 480 and deflate the inflatable shunt 400. If the activatable material 480 introduced into or resident within the inflatable shunt 400 is a polymer or substance containing nanotubes (for example, one that is initially in a liquid state), it may be activated or cross-linked by exposing it to light that causes it to transform into a gel or some more dense material. Subsequently, the activatable material 480 may be re-liquefied or changed into some other more easily withdrawable condition by, for example, again exposing it to light. This may be done, for example, via an optical fiber placed inside the lumen of the inflatable shunt 400 or in the vicinity of (e.g., adjacent to) the shunt. Depending on the activatable material 480, changes in the characteristics of the material may be achieved by exposing it to various wavelengths of light, changes in temperature or pressure, exposure to chemical substances, or other material or energy transport processes, that cause activation or deactivation of the activatable material 480 for the purpose of selectably inflating or deflating the inflatable shunt 400.

The central connecting section 430 may optionally be comprised of an inflatable element, or it may be constructed from a material and with a design that is not inflatable, e.g., a tube, a sheet, or a film. In embodiments where the central connecting section 430 is inflatable, it may optionally be configured to expand radially when it is inflated. Further, the degree of radial expansion may be selectable and related to the quantity of, or optionally the pressure of, the inflating material 480 contained within. Additionally, depending on the characteristics of the inflating material (i.e., inflatability or how much inflation the inflating material will achieve), the amount of radial expansion can also be controlled. In this way, the central connecting section 430 can expand to fill and seal the passageway through the tissue walls 450, 460. This reduces the chances that material may leak out of the connected lumens around the inflatable shunt 400, and into the space external to either lumen. The central connecting section 430 of the inflatable shunt 400 may further be configured to expand forcefully as the internal pressure or volume is increased, so that it is capable of dilating the adjacent tissue 450, 460 surrounding it. In this way, it may be used as a dilation balloon, in addition to its other functions described herein.

Further, the central connecting section 430 may optionally be configured so that it may be incrementally inflated over time, which may be used, for example, to progressively dilate a tract through lumen wall(s) 450, 460 by selectively controlling the pressure inside at least the central connecting section 430. For example, the device may be configured so that the outer diameter of the central connecting section 430 is 10 mm when it is inflated to a pressure of 1 atm, 12 mm when it is inflated to 1.5 atm, and 14 mm when it is inflated to 2 atm. In this way, the degree and forcefulness of dilation may be selected and adjusted. Further, the dilation may be selected and adjusted during a single procedure, or over a longer period of time, such as multiple procedures which may be separated by days, weeks, or months. This would allow for the progressive dilation of a tract from an initially small value (e.g., 10 mm) to a large value (e.g., 15 mm, 20 mm) that would not be possible to achieve at a single point in time or over a short time duration (e.g., minutes or hours) without risking damage to the tissue.

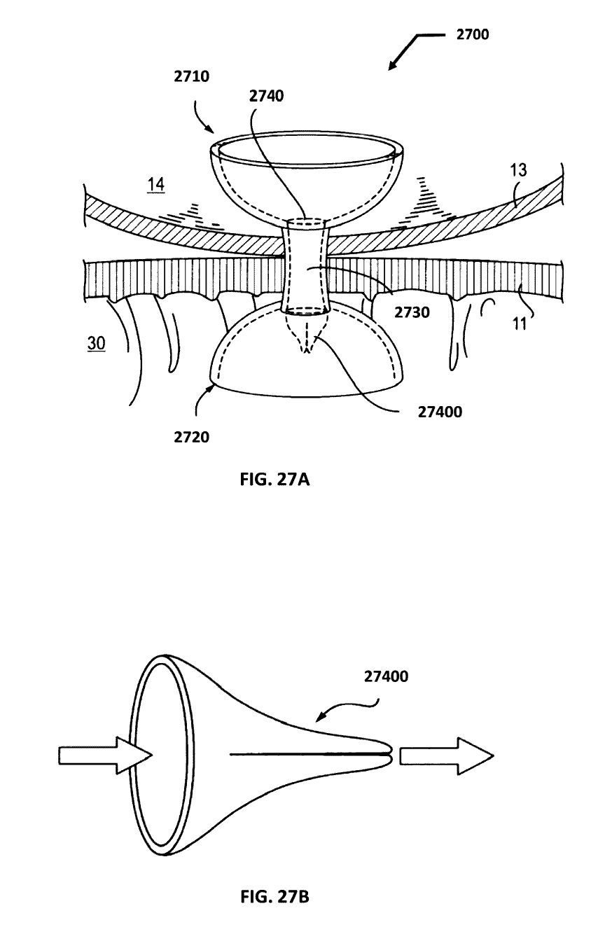

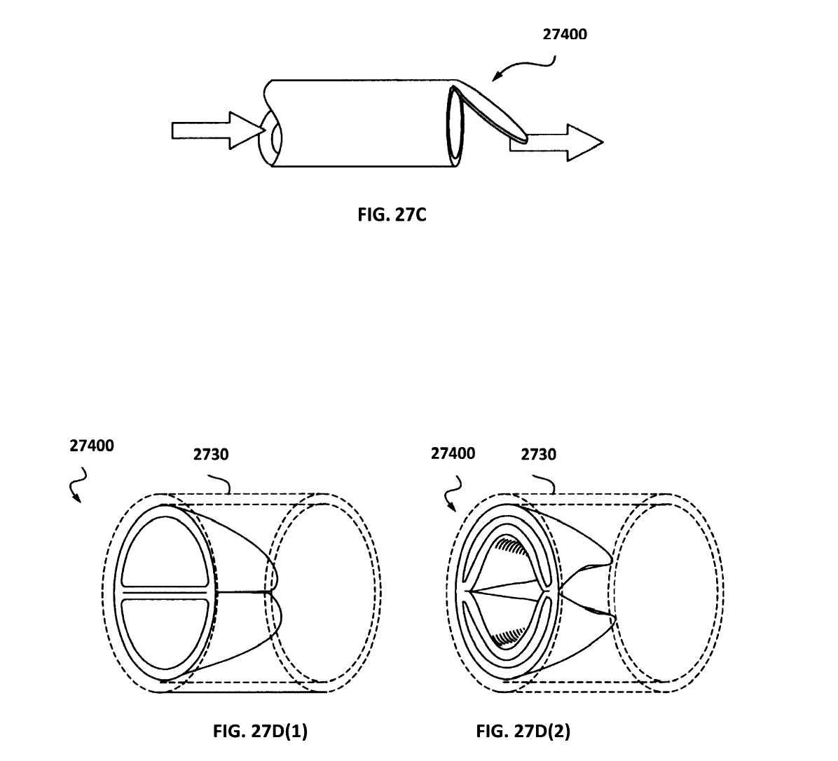

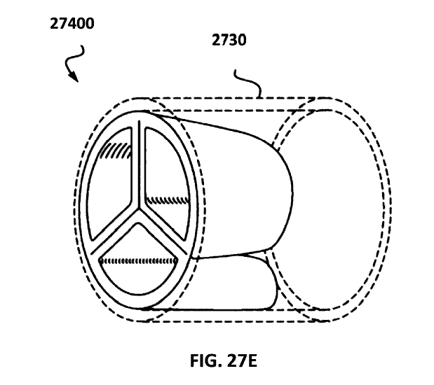

Turning now to FIG. 27A, to ensure that material is only allowed to travel through the device 2700, having a first inflatable anchor 2710 and a second inflatable anchor 2720, in a single direction (e.g., from a first lumen such as a gallbladder 14 through lumen wall 13, into a second lumen such as a duodenum 30 through lumen wall 11) and not in the reverse direction, a valve 27400 may be incorporated into or attached onto the device 2700. For instance, it may be desirable to deliver bile into the digestive tract, and to drain pus or other fluids, but undesirable for partially digested food to move from the small intestine into the gallbladder 14. A valve 27400 may be used to prevent flow into the gallbladder 14 from sources other than the biliary system. Among the purposes of the valve: allow the movement of material from a first lumen to a second lumen (e.g., gallbladder 14 to duodenum 30), prevent flow in the reverse direction (e.g., duodenum 30 to gallbladder 14), and control the level of pressure that is allowed to develop between the connected lumens. Controlling the pressure within the upstream lumen (e.g., the gallbladder 14), and ensuring that the pressure differential between the connected lumens remains low, reduces the risk of the contents of either lumen leaking into surrounding regions (e.g., bile leaking into the peritoneum). The valve may be incorporated or added at any location within the device 2700, from the most distal end to the most proximal end, or at any point within the central connecting section 2730. The valve may be made of any suitable material, such as the materials used to construct the device 2700, or unlike materials that are better suited to the function of the valve (e.g., silicone, urethane, and the like). A suitable material for this purpose is silicone. However, it may also be made of any other suitably biocompatible compliant material (in cases where bending characteristics of the material are used to create the flow control feature) or non-compliant material such as stainless steel, nickel-titanium alloy, or titanium (in cases where mechanical elements are used to create the flow control feature(s)). In one embodiment a duckbill valve is used, illustrated in FIG. 27B, with a slit in a cone-shaped compliant material that supplies elastic forces that cause it to be closed under normal circumstances, open when the pressure inside the upstream lumen (e.g., gallbladder 14) exceeds the pressure in the connected downstream lumen (e.g., duodenum 30) by a desired amount (e.g., during contraction of the gallbladder 14), and closed if the pressure in the connected downstream lumen (e.g., duodenum 30) is higher than the pressure in the upstream lumen (e.g., gallbladder 14), a condition that might otherwise result in flow in the undesirable direction). In an alternate embodiment, the valve may be designed as a sprung flap shown in FIG. 27C, with functional characteristics similar to those of the duckbill valve described above. Another embodiment is that of a bicuspid valve (closed and not allowing flow in and FIGS. 27D(1); open and allowing flow in the desired direction in and FIG. 27D(2)) or tricuspid valve (FIG. 27E), which also exhibits the desired characteristics described above. Each of these embodiments, and other valve embodiments not specifically described herein, share the flow control characteristics of a) allowing flow from a first lumen (e.g., gallbladder 14) into a connected downstream lumen (e.g., duodenum 30) through the device 2700 when the pressure in the first lumen (e.g., gallbladder 14) is greater than the pressure in the connected downstream lumen (e.g., duodenum 30) by a desired amount, b) not allowing flow from the downstream connected lumen (e.g., duodenum 30) into the first, upstream lumen (e.g., gallbladder 14) when the pressure in the downstream lumen (e.g., duodenum 30) exceeds the pressure in the upstream lumen (e.g., gallbladder 14), and c) not allowing flow from the downstream connected lumen (e.g., duodenum 30) into the first, upstream lumen (e.g., gallbladder 14) when little or no pressure differential exists between the two lumens.

A valve 27400 which is adjustable may also be incorporated into or added to a device 2700. Such a valve 27400 would enable practitioners or patients to adjust the difference in pressure between the connected lumens (e.g., the gallbladder 14 and duodenum 30) at which the valve 27400 opens. Adjustability may be incorporated into the valve body in such a way that a clinician may adjust it endoscopically, or it may be incorporated in such a way that a clinician or a patient may adjust the valve 27400 without requiring additional endoscopy or invasive procedure.

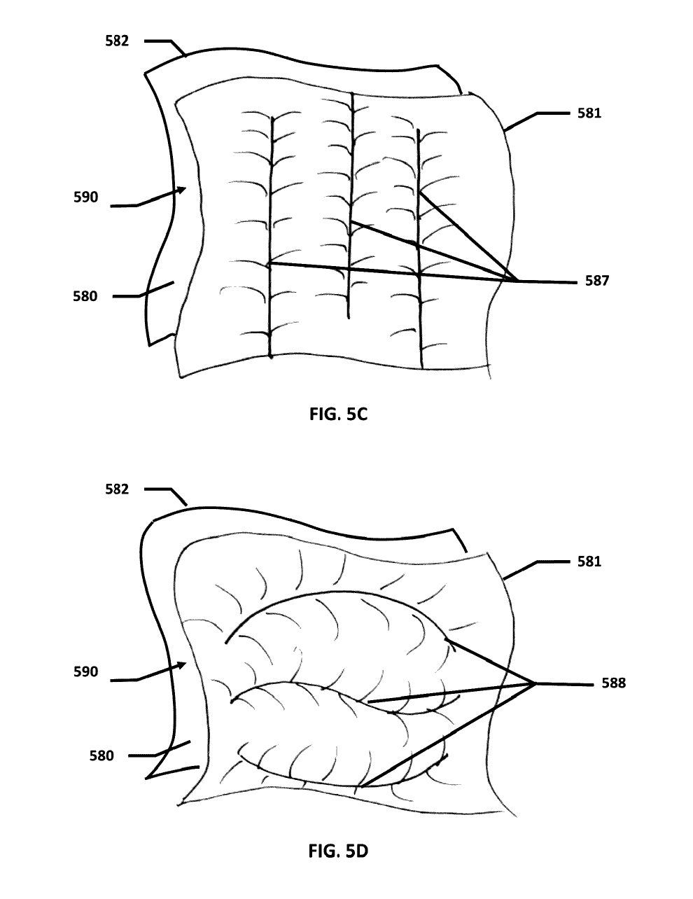

FIG. 5A-FIG. 5F illustrate features that enable the creation and maintenance of complex desired shapes of the device 500. As inflation material 580 is introduced into an inflatable region 590 or activated and, in some cases, internal pressure is created and increased, internal connections between walls may be incorporated, such as seam (see, spot or seam connection 585 in FIG. 5A, linear seam connections 587 in FIG. 5C, curved seam connections 588 in FIG. 5D, spiral seam connections 589 in FIG. 5E positioned between a first inflatable anchor 510 and a second inflatable anchor 520 on a connecting section 530, and spiral seam connections on a flat section 591 in FIG. 5F) or spot connections (e.g., welds 586 shown in FIG. 5B), between a first wall of the inflatable elements 581 and a second wall of the inflatable elements 582 of the device 500. It should also be appreciated that more than two walls may be joined using this approach. By joining layers at strategically selected points, (e.g., “spot welds” 585 in FIG. 5A and spot connections 586 in FIG. 5B and arrangements or arrays of any linear or curved connections (e.g., “seam welds”); areas (e.g. “region welds”), or combinations of any or all of the various internal connections), the internally increasing volume, pressure, or stiffness of the inflation material 580 will cause the first wall of the inflatable elements 581 and the second wall of the inflatable elements 582 (as well as additional walls, if included in the connections between walls) of the devices 500 to create and maintain a desired shape. Additionally, the thickness of the walls of the inflatable elements 581, 582, and/or other wall material properties, such as stiffness, elasticity, durometer, and the like, may be selected to differ in selected regions of the inflatable region 590, enabling them to achieve a desired configuration and/or shape when inflated.

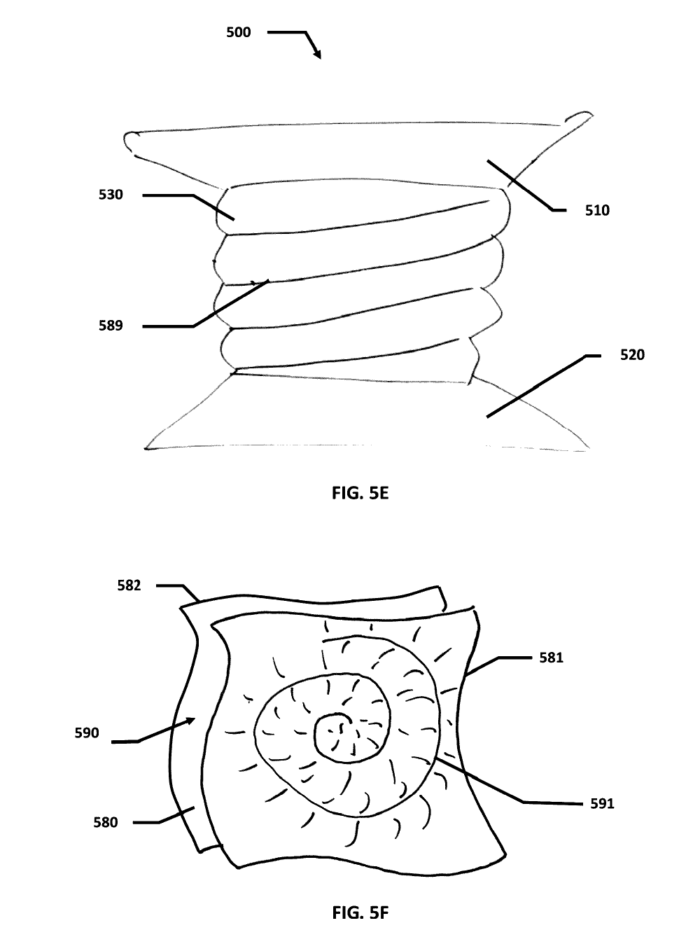

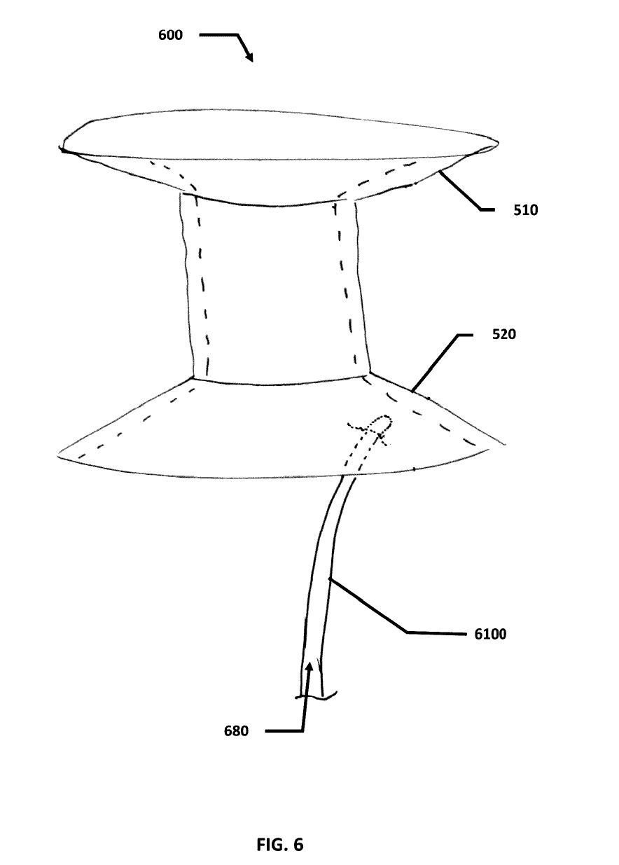

In cases where the shunt device 600 shown in FIG. 6 is inflated during a procedure by introduction or injection of a inflation material 680 (e.g., fluid, gas, activation component) that causes the inflatable shunt device 600 having a first inflatable anchor 510 and a second inflatable anchor 520, to assume its final or deployed configuration (e.g., by expansion), an inflation element 6100, such as a tube, may be used that introduces or enables the activation of the inflation material 680. The inflation material 680 that causes the inflation of the shunt (e.g., fluid, gas, activation component) may be introduced by a variety of inflation elements 6100, e.g., a tube or syringe, when the material is a liquid or a gas, or by a mechanism that grips, repositions, pushes, crushes, mixes, etc., in cases where solids (which may be comprised of a large number of small solid elements, such as grains or powders), liquids, gases, or mixtures of solids, liquids, or gases are introduced, repositioned, or reconfigured, or by an optical fiber, in cases where a material is caused to transition when illuminated with light energy between a small volume and a large volume, or a low viscosity to a high viscosity, or from a fluid to a solid.

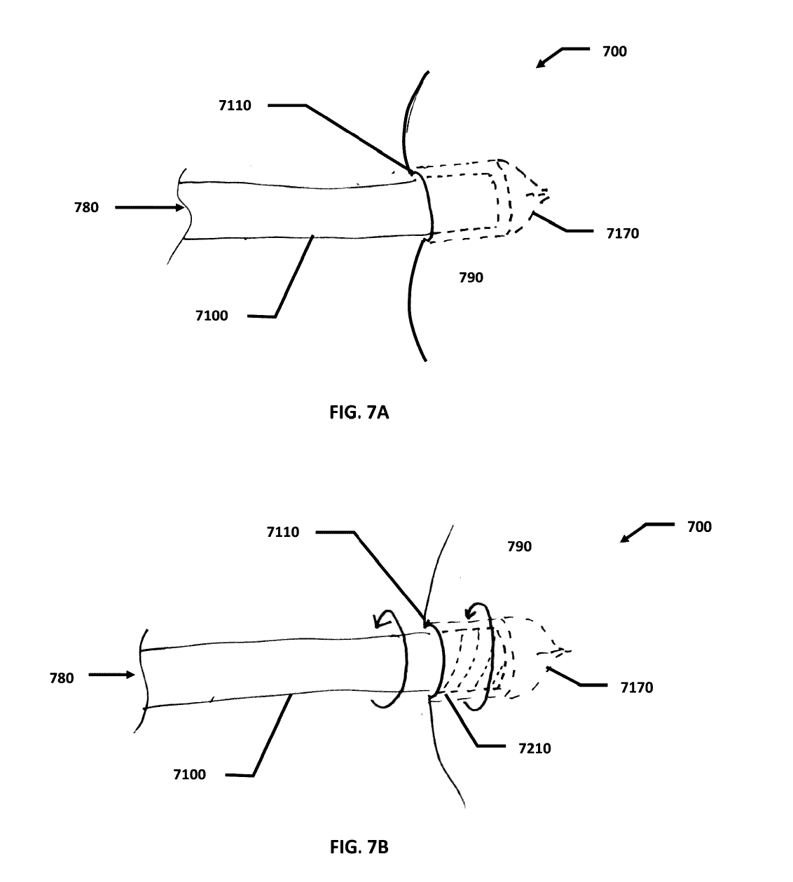

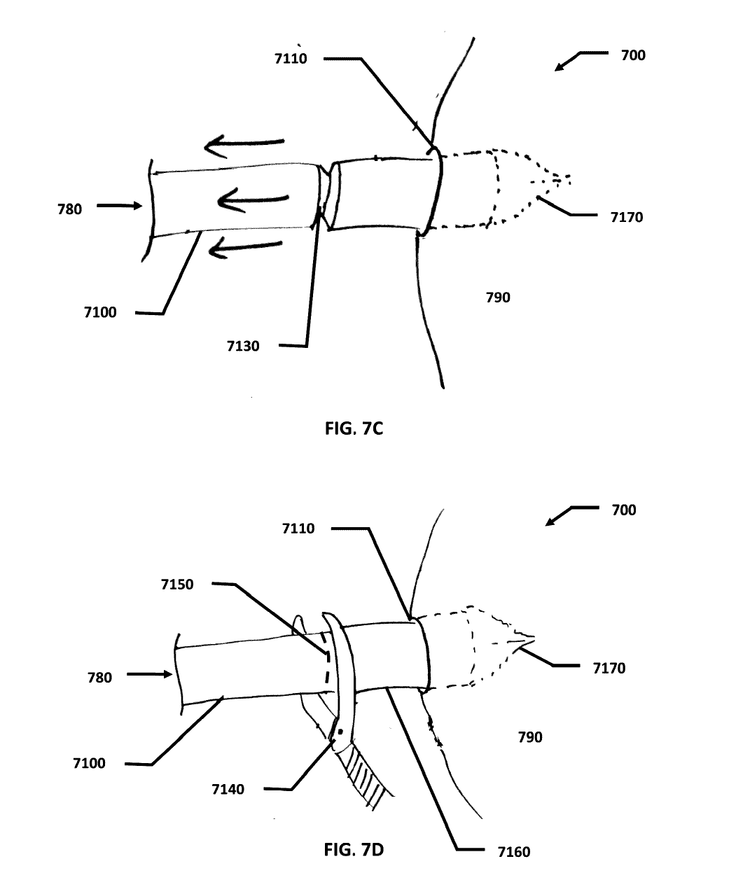

The inflation element 6100 may optionally be disconnected from the inflatable shunt device 600 at some time during a procedure. For example, once the inflatable shunt device 600 is inflated and caused to assume its final or deployed configuration, the inflation element 6100 may be detached from the inflatable shunt device 600 and withdrawn from the body of the patient. This may be achieved in a variety of ways. For example, in an embodiment shown in FIG. 7A, an inflation element (such as a tube) 7100 is used to introduce an inflation material 780 into an inflatable region 790 within an inflatable shunt 700, the inflation element 7100 may be secured to the inflation port 7110 by use of a friction fit/seal. In such an embodiment, the inflation element 7100 may be removed from the inflation port 7110 by applying tension to the inflation element 7100 to slide it out of the inflation port 7110. Alternatively as shown in FIG. 7B, a twist-lock or threading feature 7120 may be used to engage the inflation element 7100 and the inflation port 7110, in which the inflation element 7100 may be rotated, preferably counterclockwise, to detach it from the inflation port 7110.

Yet another alternate approach to detaching from the inflatable shunt FIG. 7C involves severing the inflation element 7100. For example, once inflation is complete, applying sufficient tension or torque to a break-away section of an inflation tube 7130 may cause it to detach from the inflatable shunt 700. Alternatively as shown in FIG. 7D, a cutting device 7140 may be used to cut an inflation element 7100 at a desired location 7150, leaving a portion 7160 (which may be very small, or even flush with the wall of the inflatable shunt 700) of the inflation element attached to the inflatable shunt 700.

Prior to detaching the inflation element 7100, in some embodiments shown in FIG. 7A-7D it may be beneficial to seal the inflation port 7110 or section of inflation element 7100 (e.g., proximal to the inflation port 7110). In an embodiment, the inflation material 780 is a fluid (e.g., saline), and the inflation port 7110 is a small diameter (e.g., about 1 mm) tube. For this embodiment, a small one-way flow control element 7170, such as one-way valve (e.g., duck-bill valve, ball valve, flap valve, sleeve valve) may be incorporated into the inflatable shunt 700 or inflation port 7110 that allows inflation material 780 to flow into an inflatable region 790 of the inflatable shunt 700, but does not allow reverse flow of the inflation material 780, out of the inflation port 7110. In this case, once the inflatable shunt 700 has been inflated through the inflation port 7110, the inflation element 7100 (e.g., a tube) may be removed, and the inflation material 780 (e.g., saline) will remain inside the inflatable region 790 of the inflatable shunt 700, which then retains the desired shape and characteristics. In one example, shown in FIG. 7B, the inflation element 7100 can be removed from the inflation port 7110 by rotating the inflation element 7100 (e.g., releasing via the threads 7210).

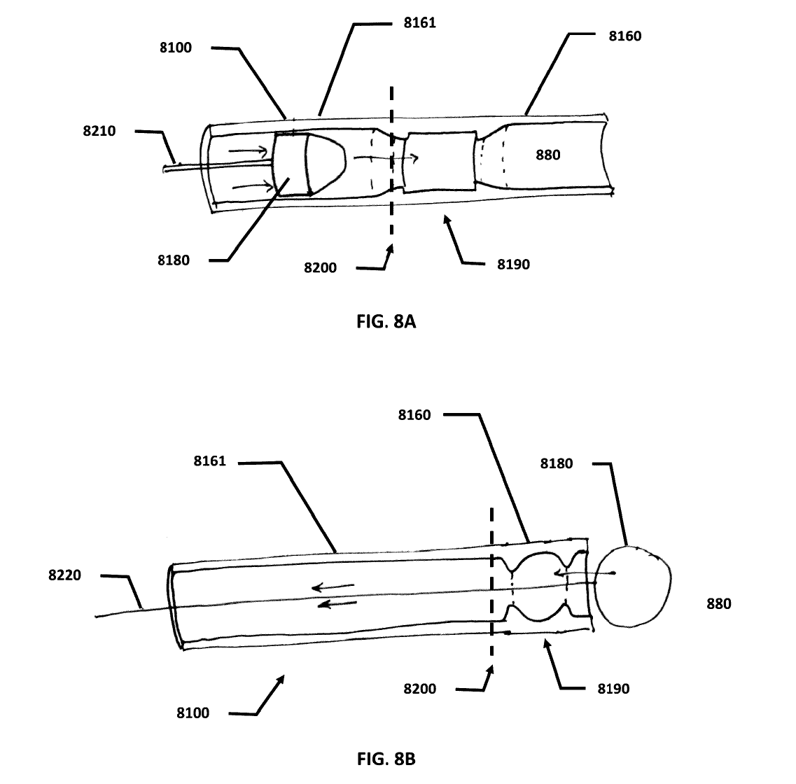

In still other embodiments (FIG. 8A, FIG. 8B), retention of the inflation material 880 may be achieved by eliminating flow within the inflation element 8100 (e.g., by closing off or “stopping” a tube with a stopper 8180) or the inflation port 8110 prior to disconnecting the inflation element 8100 from the inflatable shunt. The location of the stoppage 8190 of the closure or stoppage of the inflation element 8100 may be selected so that the section of the element containing the stoppage 8190, and optionally a portion of the inflation element 8160 remains attached to the inflatable shunt after a removable portion 8161 of the inflation element 8100 is detached at a detachment location 8200 and removed. In this way, the inflation material 880 remains inside the shunt and the shunt maintains the deployed or final configuration. This may be achieved, for example, by introducing a stopper 8180 into a mating and/or retaining feature within the inflation element 8100 (e.g., tube), the inflation port, or the inflatable region of the inflatable shunt. The stopper 8180 may be positioned, for example, by pushing it into position with a wire 8210, or, alternatively, by pulling it into position with a wire 8220 shown in FIG. 8B. The stopper 8180 may be any suitable shape, such as a bullet-shape in FIG. 8A, a ball in FIG. 8B, and a cone (with or without rounded edges). Once the stopper 8180 has been positioned in the desired location of the stoppage 8190 to prevent the flow of inflation material 880, for example, a location of the stoppage 8190 with a mating feature that conforms to and presses around the exterior of the stopper 8180, such as a notch or a region with a reduced inner diameter, the stopper 8180 will prevent flow within the inflation element 8100 in either direction.

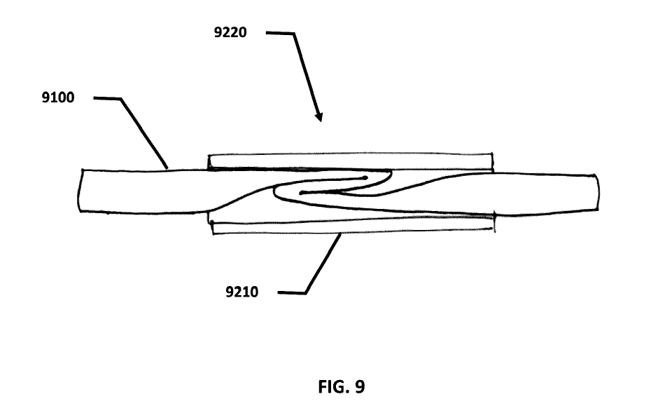

In another embodiment shown in FIG. 9, the inflation element 9100 (e.g., a tube) may be folded, kinked, or otherwise clamped shut at a desired location 9220. Once the inner lumen of the inflation element 9100 has been closed, an additional component such as a collar 9210 may be introduced to maintain this condition. For example, in cases where the inflation element 9100 comprises a tube and it has been kinked to cause it to close 9220, a collar 9210 may be positioned over the kinked portion, locking it in this condition indefinitely, or until the collar 9210 is removed.

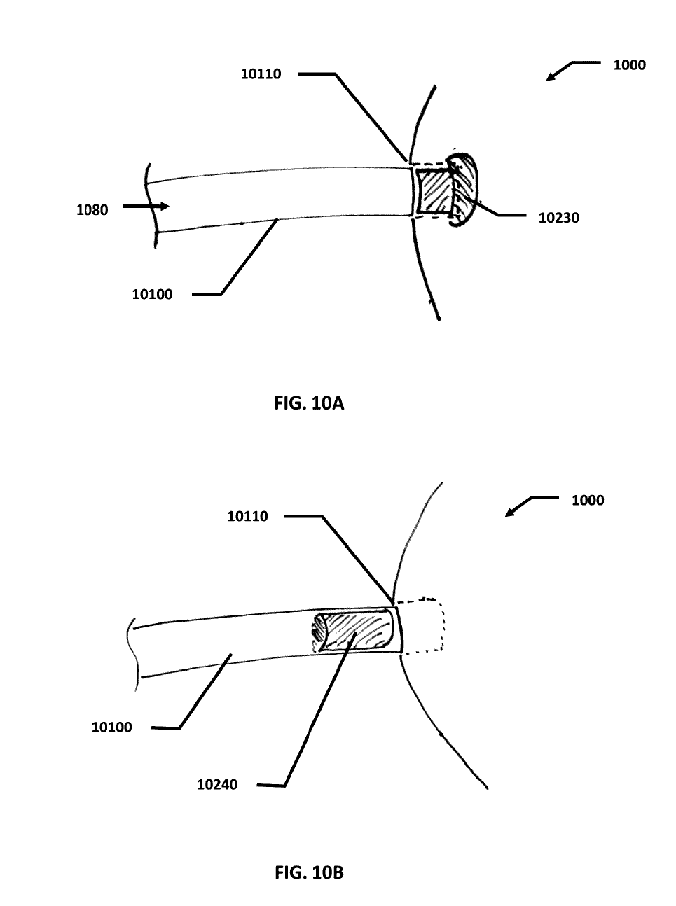

In yet another embodiment FIG. 10A, the inflation port 10110 may be sealed with a material 10230, such as an adhesive, for which a desirable characteristic is that it transitions from an initially deliverable configuration (e.g., a liquid), may be activated (e.g., via exposure to ultra-violet light, oxygen, or the absence of oxygen), and subsequently transforms into a material with a final configuration that remains in position (e.g., a solid or near-solid which fills or blocks the port, and, in some cases, adheres or bonds to the adjacent surface(s)) and prevents the flow of inflation material 1080 into or out of the shunt device 1000. This blockage may likewise be selectably positioned within the inflation element 10100 shown in FIG. 10B. In some configurations, the inflation element 10100 comprises a tube, and the blocking material 10240 may be selectably positioned along some portion or all of the length of the inflation element 10100. Any suitable type of adhesive or other activatable material may be used to create a blockage from the blocking material 10240 in the inflation element 10100, or the inflation port 10110 provided it seals the inflation element 10100, inflation port 10110, or inflatable region of the shunt device 1000, at a desired time, in a desired location, and under the control of the user.

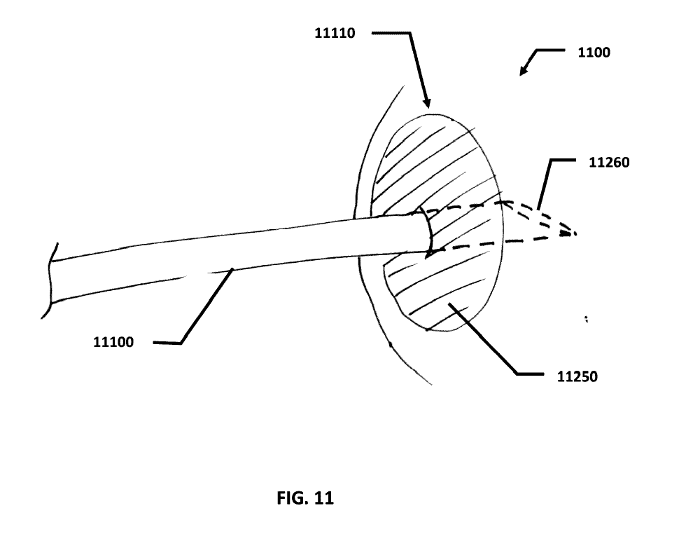

FIG. 11 illustrates at least the inflation port 11110 comprises a self-healing region of material 11250 (e.g., a low durometer silicone) which may be incorporated into the wall of the inflatable shunt 1100 (or make up a portion of the inflatable shunt 1100, or the entire inflatable shunt 1100) and designated as the site through which a sharp inflation tip 11260 at a distal end of an elongated tubular member 11100 may be inserted to introduce inflation material(s). This may be done, for example, though a hypodermic needle or a fine needle aspiration (FNA) needle. Further, the self-healing region may be formulated to be radio-opaque, echogenic, or otherwise designed to be easily distinguishable from other proximal materials, so that the user may accurately guide the inflation tip 11260 to the desired target.

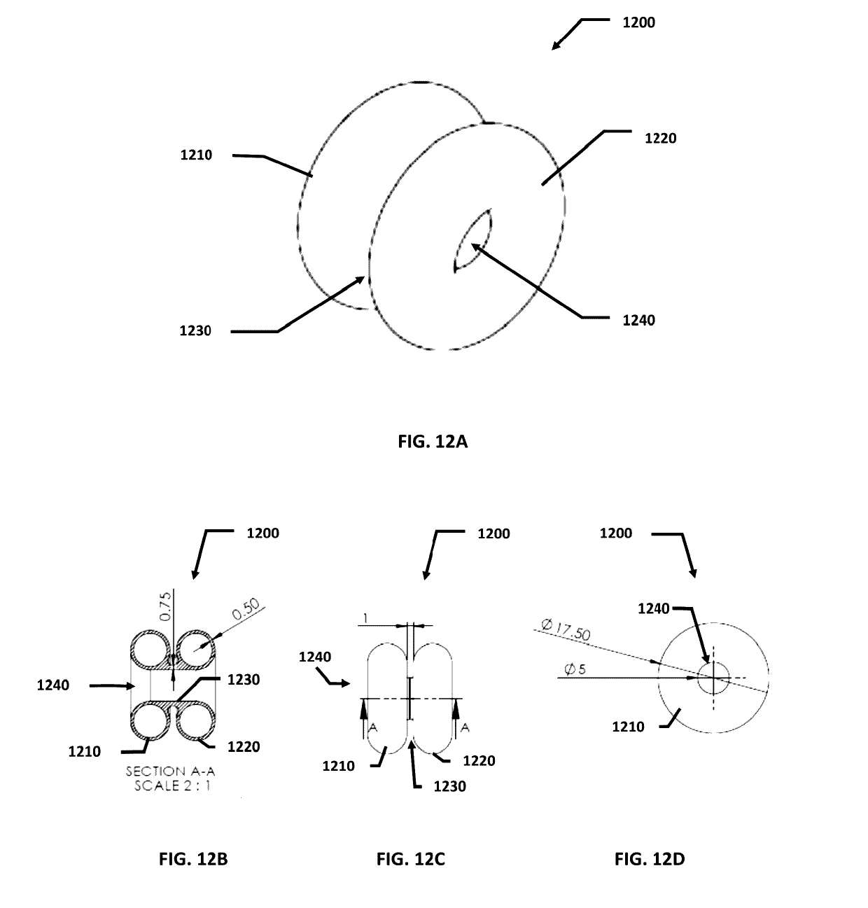

A wide variety of geometries and configurations may be used for the inflatable shunt. In addition to the embodiment comprising inflatable disc-shaped anchors FIG. 3, another embodiment FIG. 12A-12D illustrates a configuration that incorporates one or more anchors 1210, 1220, that are toroidal donut-shaped inflatable/deflatable anchors, on either end of a central connecting section 1230. As with previously described embodiments, such a variation may incorporate a central connecting section 1230 that may be inflatable or non-inflatable. The length of the central connecting section 1230 may be varied, depending on the procedure and the specific anatomy of the patient. A central aperture 1240 may be provided. The dimensions shown in FIG. 12B and FIG. 12D are representative, and the suitable range of dimensions may vary substantially from those shown. In some cases (e.g., when the gallbladder is connected to the duodenum with the shunt 1200), the length of the central connecting section 1230 may be selected so that the anchors 1210, 1220 are relatively close together, e.g., 1-10 mm. In other cases (e.g., when the gallbladder is connected to the stomach with the shunt 1200), the length of the central connecting section 1230 may be selected so that the anchors are further apart, e.g., 5-15 mm, or more.

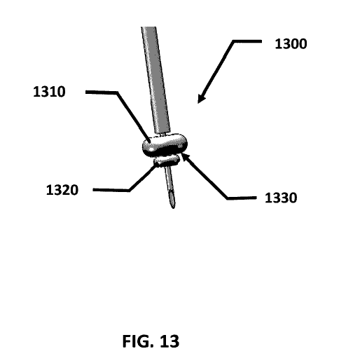

In a variation of the configuration of FIG. 13, the anchors 1310, 1320 are positioned on either side of a connecting member 1330 and have different sizes (e.g., one or both of height and diameter). By altering the size of the anchors 1310, 1320 (e.g., differing inner diameters, outer diameters, and the like), the location of the optional application of forces to the tissue held between the anchors may be selected, as discussed above relative to FIG. 4. In cases where pressure is applied to the tissue held between the anchors, with the goal of causing pressure necrosis and eventual detachment of the necrosed tissue, the anchors 1310, 1320 shown in FIG. 13 are configurable so that the smaller anchor 1320 is initially placed within the lumen from which the shunt 1300 is to move out of (e.g., the gallbladder) and the larger anchor 1310 placed within the lumen into which the shunt 1300 is to move into after detaching (e.g., the duodenum). The shunt 1300 will move out of the lumen which contains the smaller anchor 1320 and into the lumen that contains the larger anchor 1310 once pressure necrosis occurs. This characteristic and effect may be exploited for any of the configurations of inflatable shunt described herein: each may include a larger and a smaller anchor, and the direction of motion of the shunt upon the occurrence of pressure necrosis thereby selected.

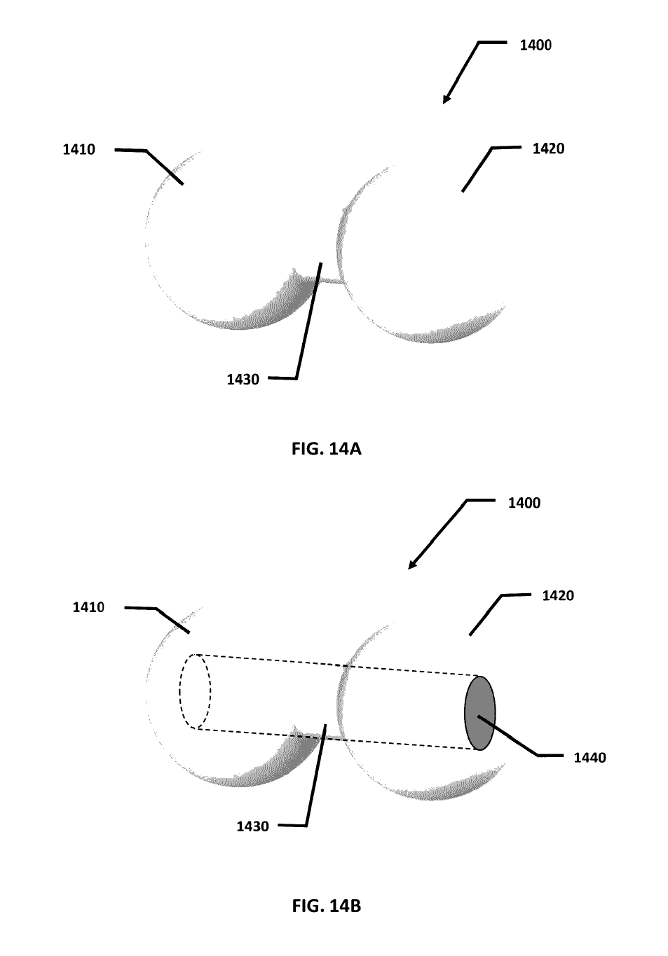

Other embodiments involve the incorporation of at least one spherical, or near-spherical, anchor as shown in FIG. 14A-B. In one case, spherical anchors 1410, 1420 are incorporated on both ends of a central connecting section 1430, the spherical anchors 1410, 1420 and the central connecting section 1430 optionally incorporate a lumen 1440 through the device 1400, connecting the distal end of the spherical anchor 1410 is distal to the proximal end of the spherical anchor 1420.

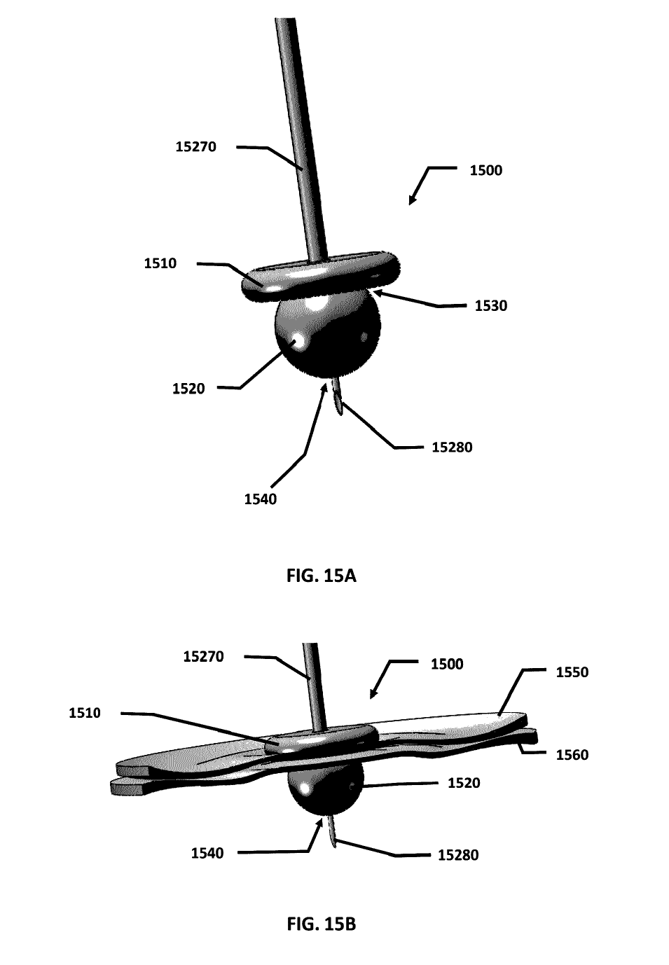

Still other embodiments involve different types of anchors. In a variation of an embodiment of an inflatable shunt as shown in FIG. 15A-B, the proximal anchor 1510 is configured as a toroid, and the distal anchor 1520 is configured as a round or spherical shape. An optional inner lumen 1540 may optionally be included through the center of the anchors to allow for the passage of material and/or tools. In this configuration, the spherically shaped distal anchor 1520 may be placed in the distal lumen, through the lumen wall 1560 (e.g., the gallbladder) and the toroidal balloon forming the proximal anchor 1510 placed in the proximal lumen, proximal to the lumen wall 1550 (e.g., the duodenum). The optional inner lumen 1540 of the inflatable shunt device 1500 creates a communication between the distal and the proximal lumen. Further, the optional pressure applied to the tissue of the lumen walls 1550, 1560 (as discussed above relative to FIG. 4) may be selected to be sufficient for the creation of pressure necrosis, after which the necrosed tissue will detach from the healthy surrounding tissue, carrying the inflatable shunt device 1500 away with it. In this configuration, the outer diameter of the toroidal balloon forming the proximal anchor 1510 is larger than the spherically shaped distal anchor 1520, so the inflatable shunt device 1500 and tissue will move in the direction of the toroidal balloon forming the proximal anchor 1510, into the more proximal lumen (e.g., the duodenum), where it can be cleared from the body by a natural process, such as peristalsis. The inflatable shunt device 1500 is depicted positioned on an elongated tubular member 15270 with a sharp distal end 15280 suitable for piercing.

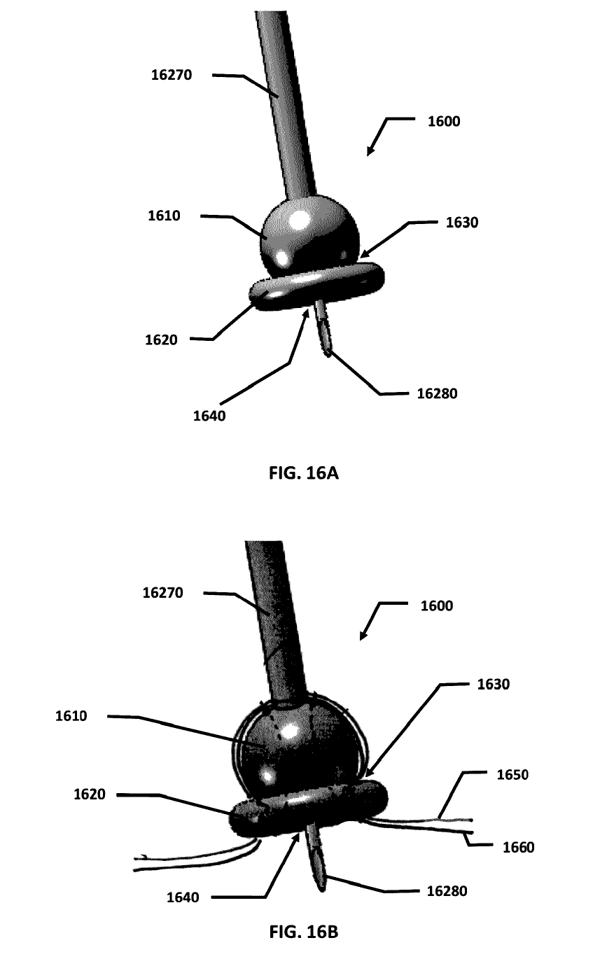

Another variation is shown in FIGS. 16A-B which involves exchanging the position of the spherical anchor 1610 and the toroidal anchor 1620. In this variation, the spherical anchor 1610 is again delivered to the distal lumen (e.g., the gallbladder) through the first lumen wall 1650 and the second lumen wall 1660, and the toroidal anchor 1620 in the proximal lumen (e.g., the duodenum). However for this variation the tissue of the first lumen wall 1650 and the second lumen wall 1660 are pulled back around and over the spherical anchor 1610 and secured in this position by the toroidal anchor 1620, which acts as a ring around the spherical anchor 1610. The spherical anchor 1610 is configured to be larger in diameter when inflated than the inner diameter of the toroidal anchor 1620 when inflated so that the spherical anchor 1610 is prevented from pulling through, and thus clamps the first lumen wall 1650 and the second lumen wall 1660 of the connected lumens in place. Substantial clamping force, and thus pressure, may be created and imparted to the tissue of the first lumen wall 1650 and the second lumen wall 1660 held between the spherical anchor 1610 and the toroidal anchor 1620, and this may be selected to be sufficiently high that pressure necrosis results (as discussed above relative to FIG. 4). Again, since the spherical anchor 1610 is smaller within the distal lumen (e.g., the gallbladder), and the toroidal anchor 1620 is larger within the proximal lumen (e.g., the duodenum), the necrosed tissue and inflatable shunt 1600 will pass into the proximal lumen upon detachment. As with the subsequently described embodiment (below), a layer of material, or connecting component (e.g., a sleeve, a net, a mesh, or more than one cord, thread, ribbon, or strip) may be used to attach the spherical anchor 1610 to the toroidal anchor 1620, e.g., through the central connection component and/or optional inner lumen 1640, however in the embodiment illustrated, one end of the connecting component 1630 is attached to the proximal end of the spherical anchor 1610 and is arranged along the inside of the spherical anchor 1610, forming an inner lumen. The other end of the material is attached to the inner diameter of the toroidal anchor 1620. When both the spherical anchor 1610 and the toroidal anchor 1620 are inflated, the connecting component transmits tension forces and pulls the toroidal anchor 1620 snugly against the spherical anchor 1610, securely clamping the anchors together around the lumen wall tissue, and the clamping pressure may optionally be selected to be sufficient to cause pressure necrosis. Other arrangements for the connecting component are possible, such as positioning it so that it connects to the proximal end of the spherical anchor 1610 as before, but is routed along the outer surface of the sphere, connecting to the inner diameter of the toroidal anchor 1620. The inflatable shunt device 1600 is depicted positioned on an elongated tubular member 16270 with a sharp distal end 16280 suitable for piercing.

In order to generate and impart more substantial clamping pressure to the first lumen wall 1650 and the second lumen wall 1660, a connecting component or components comprised of materials dissimilar to and less flexible than the materials used to construct the inflatable/deflatable balloon elements of the inflatable shunt 1600 may be incorporated (such as a sleeve, a net, a mesh, or more than one cord, thread, ribbon, or strip) that either passes though, or serves as, the connecting component 1630 which can be centrally positioned, or, alternately, an inner lumen. The connecting component 1630 may be attached on one end at the furthermost point of the spherical anchor 1610 and on the other end to the inner diameter of the toroidal anchor 1620, with the length of the connecting 1630 component running through the center of the spherical anchor 1610 and the center of the toroidal anchor 1620. In this way, inflation of either anchor will serve to apply tension and hence clamping forces between the anchors, transmitted by the connecting component.

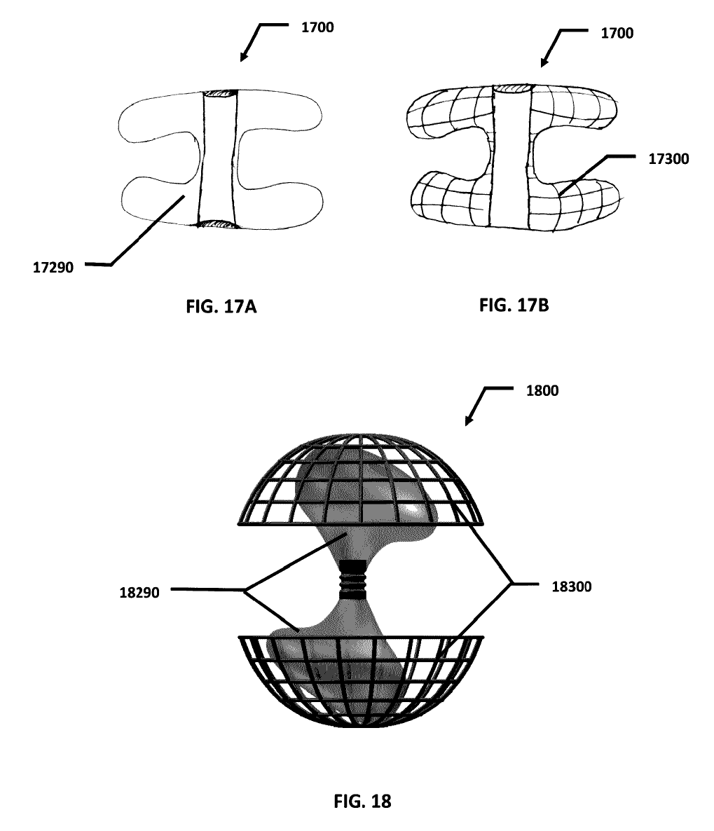

As shown in FIGS. 17A-B an inflatable shunt 1700 is one in which there are two primary components: an inflatable component 17290 and a structural component 17300. The inflatable component 17290 is constrained by the structural component 17300 (for example, the inflatable component 17290 may fill a cavity defined by the structural component 17300), and the structural component 17300 will define the desired shape and cause the inflatable component 17290 to conform to that shape.

Turning to FIG. 18, a device 1800 is shown with an inflatable component 18290 and one or more structural components 18300 involves containing the inflatable component 18290 within the structural component 18300, such as an outer sheath, housing, or covering. This provides structure and constrains the anchoring flanges so that they take on the desired shape once inflated. In one example of this variation, the structural component 18300 defines a concave space, and as the anchors of the inflatable component 18290 inflate, they initially occupy and fill the concave space within the structural component 18300, and once the available volume has been filled, further inflation of the anchors causes them to expand inward toward each other, resulting in the application of pressure to the tissue held between the anchoring balloons. The structural component 18300 may be rigid, semi-rigid, or flexible, and collapsible to a small profile for delivery before the balloons are inflated within them. The structural component 18300 may be integrally formed with the surface of the inflatable component 18290 (e.g., formed with thicker sections of balloon material, co-molded with disparate materials, encapsulated or bonded Nitinol wire, stainless steel, spring metal, other polymers, etc.), or they may be separate components (e.g., baskets as illustrated in FIG. 18, which may be comprised of polymers, shape metals like Nitinol, stainless steel, etc.). The structural component 18300 may be held a fixed distance away from each other, so that as the inflatable component's 18290 anchor balloons inflate and expand inward, the structural component 18300 do not move away from the tissue. In this case, they provide reaction forces that resist the forces created by the inflatable anchors as they inflate. This may be achieved through the use of a component that fixes the distance between the structural elements, such as a wire or tube attached to either structural element which sets the maximum spacing between them, and resists loads that would otherwise push them apart.

An aspect of an inflatable shunt is how its profile (cross-sectional area) is minimized for delivery into a patient. The inflatable anchoring features may be deflated to minimize their overall size, and, together with the central connecting section, the profile of the device will be further reduced, in some cases, to the point where it will fit through the tool channel of an endoscope. Typical endoscope tool channels range from about 2.8 mm to about 5 mm in diameter. One method for reducing the packed profile of the balloon shunt is to flatten at least the central connecting section and roll it into a smaller configuration.

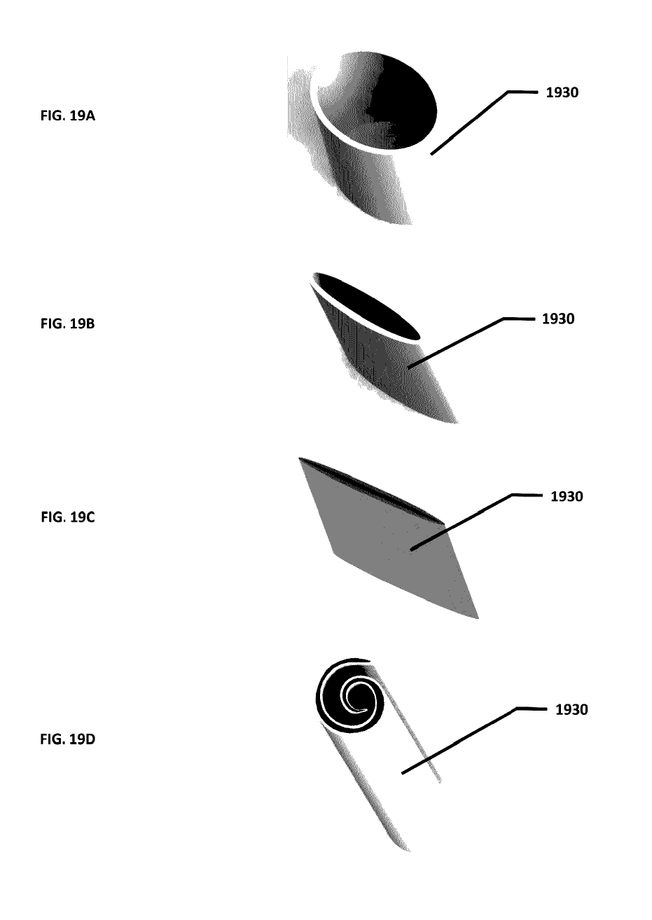

FIG. 19A shows the unconstrained initial shape of the central connecting section 1930. FIGS. 19B-C show the central connecting section 1930 being progressively flattened.

FIG. 19D shows the central connecting 1930 section rolled to further reduce its profile. The inflatable anchors at either end of the central connecting section 1930 may be similarly rolled. Further, they may be rolled around the central connecting section 1930, either prior to or subsequent to when it is rolled. Once the inflatable anchors are wrapped around the central connecting section 1930 which has been rolled, they may be used to hold the central connecting section 1930 in this configuration. Thus, elements of the design itself are employed to constrain the device when it is made compact for delivery. Alternately, the anchors they may be axially extended away from the central connecting section 1930, either by folding them over or by pulling axially at either end of the anchors, and they may then be subsequently flattened and rolled, so that their profile is minimized to the desired extent. If the central connecting section 1930 comprises a balloon, it may be deflated prior to flattening. Further, to maximally reduce the size of any balloon component within the device, vacuum may be applied to the balloon lumen(s) to evacuate and collapse it (them) further than may be achieved without actively withdrawing material under vacuum. Once evacuated, the inflation/evacuation port or inflation device may optionally be closed and kept closed during delivery, or until such time that the user desires to increase the size of the shunt.

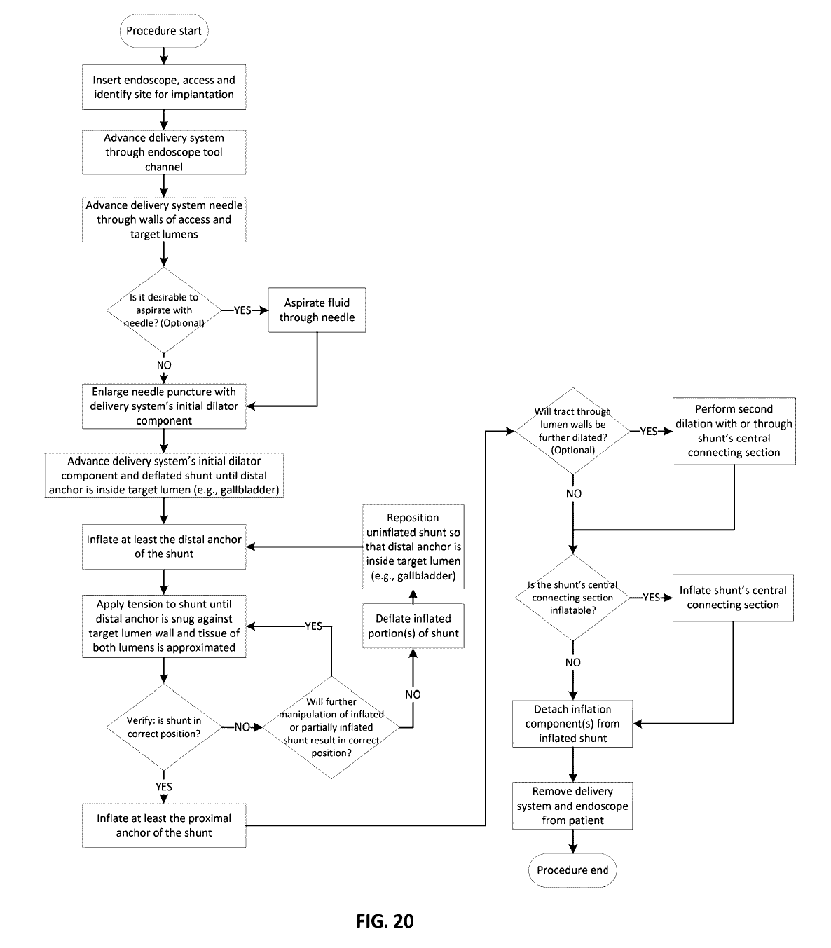

Delivery of the inflatable shunt to the desired site of implantation within the body may be achieved in a variety of ways. For example, the desired implantation site may be identified and accessed with a flexible endoscope. When an endoscope is used to access the site for shunt delivery, the site may be identified through the use of direct endoscopic visualization, endoscopic ultrasound, external ultrasound, fluoroscopy, magnetic resonance (MR) imaging, computed tomography (CT) imaging, or any combination of these or other medical imaging modalities. Preferably, endoscopic ultrasound is used because it provides visualization through the wall of the gastrointestinal tract without the use of ionizing radiation. Once the desired implantation site has been accessed and identified, the process of implanting the inflatable shunt may be initiated. An exemplar delivery method is shown in FIG. 20 which illustrates a method including: (1) using an endoscope to access and identify the desired implantation site; (2) advancing the delivery system through the tool channel of the endoscope until it emerges from the tool channel at the distal tip of the endoscope; (3) advancing a needle component of a delivery system and puncture the lumen wall tissue of the access lumen (e.g., the duodenum) and the lumen wall tissue of the target lumen (e.g., the gallbladder), so that the tip of the needle resides within the target lumen (e.g., the gallbladder); (4) optionally aspirating fluid with the needle (which may be performed in this or any subsequent step); (5) enlarging the puncture through the walls of the lumens so that they will accommodate larger components (such as an inflatable shunt) by advancing an initial dilation component of the delivery system (preferably, but not limited to, the application of RF energy to electro-surgically enlarge the puncture) through the tissue; (6) advancing the deflated shunt through the enlarged puncture, until its distal anchor is positioned inside the target lumen (e.g., gallbladder), preferentially by further advancing the initial dilation component to which the inflatable shunt is also mounted; (7) inflating at least the distal anchor of the inflatable shunt; (8) applying tension to the inflatable shunt though a positioning component (e.g., the component which positions the initial dilation component) so that the distal anchor contacts and applies tension to the inner surface of the target lumen wall (e.g., gallbladder), the central connecting section extends through both lumen walls, and the proximal anchor resides within the access lumen (e.g., duodenum); (9) verifying or ensuring the desired position of the inflatable shunt's proximal anchor inside the access lumen (e.g., duodenum), readjusting its position if necessary; (10) inflating the shunt's proximal anchor; (11) optionally performing a second dilation step, either by inflating the shunt's center connecting section or by positioning and activating a second dilation element inside the shunt's inner lumen, further opening the tract through the lumen walls; (12) inflating the shunt's center connecting section (if configured for inflation, and if not already inflated in a previous step); (13) detaching the inflation elements from the fully inflated shunt; (14) withdrawing the delivery system; and (15) withdrawing the endoscope.

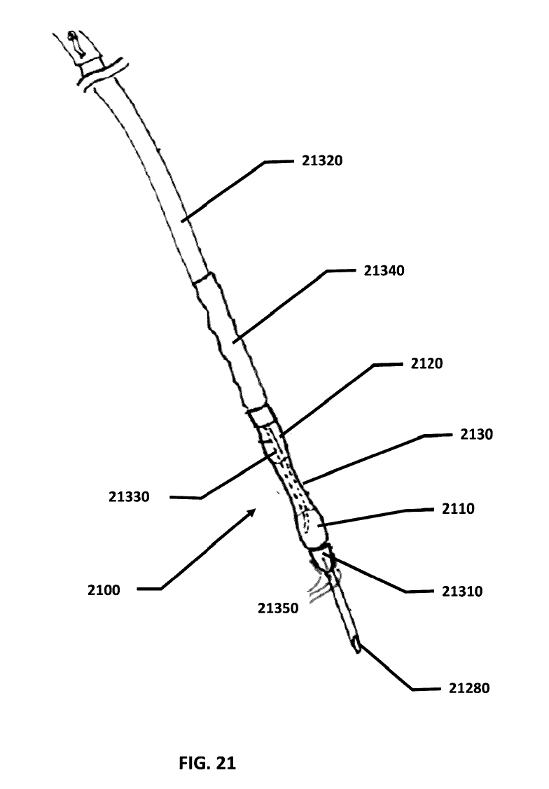

The delivery system can be configured as a single, multi-purpose tool such as that shown in FIG. 21 which incorporates all of the components for performing the necessary steps and delivering the inflatable shunt 2100. Alternatively, more than one tool may be used to deliver the inflatable shunt. Each tool may be used to perform a single step, or each tool may perform two or more steps, together comprising a kit of tools for delivering the proposed inflatable shunt.

A delivery system that combines all necessary components into a single device for delivering the inflatable shunt FIG. 21 is configured to be insertable through the tool channel of the endoscope. The inner diameter of typical endoscopic tool channels range from about 2.8 mm and 5 mm. The delivery system preferably incorporates at least some of: a needle 21280; a dilation component 21310, or cutting tool, for the initial enlargement of the needle puncture (if said dilation component is an electro-surgical component, it may incorporate one or more electrodes 21350 to advance through tissue and enlarge the puncture); an inflatable shunt 2100 in its initial/deflated configuration; an advancement mechanism 21320 that positions the dilation component 21310 and the inflatable shunt 2100 relative to the needle 21280; inflation element(s) 21330 that deliver(s) inflation material, energy, or component(s) that causes the inflation of the inflatable shunt 2100 or separately inflatable sections of a first inflatable anchor 2110 a second inflatable anchor 2120 and a connecting section 2130; an optional second dilation element for enlarging the tract through the lumen walls occupied by the shunt's central connecting section 31340, which may itself be slidably positionable relative to the inflatable shunt 2100, the dilation component 21310, and/or the needle 21280; and a handle for user controls. For a configuration of a delivery system, the needle 21280 serves as both the initial access element and the guidance element for all subsequent steps. After the needle 21280 punctures from the access lumen into the target lumen, the components that perform subsequent steps optionally slide axially over the needle shaft (e.g., the dilation component 21310, the inflatable shunt 2100, the second dilation component 21340). An optional exception to the use of the needle as the guidance element for all steps is the insertion of the second dilation component 21340, which may optionally be performed without using the needle 21280 to guide the second dilation component 21340.

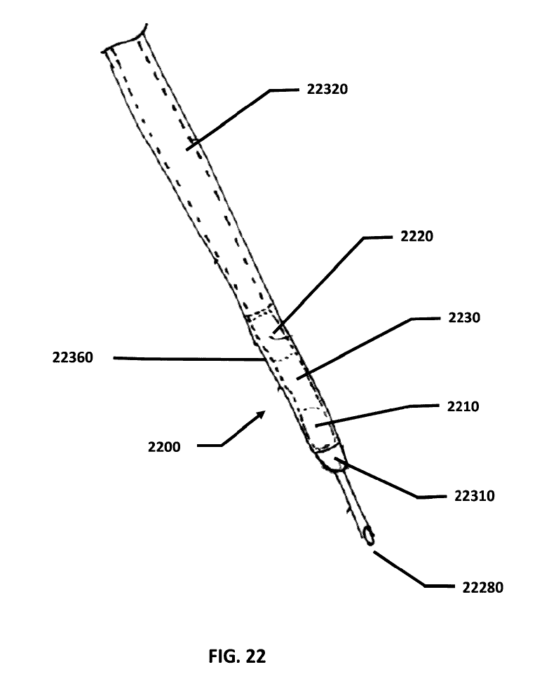

The inflatable shunt 2200 shown in FIG. 22 has a first inflatable anchor 2210, a second inflatable anchor 2220 and a connecting section 2230, and may optionally be covered by a sheath 22360 during delivery, in which case a positioning component 22320 may be incorporated that moves the sheath 22360 relative to the shunt. In a further variation, a separate advancement mechanism independently positions the inflatable shunt 2200 relative to the needle 22280, the dilation component(s) 22310, and/or the sheath 22360 for the inflatable shunt 2200, should one be used.

Preferably, the initial dilation component 21310 shown in FIG. 21 comprises an electro-surgical tool, and enlarges the puncture through the tissue made by the needle by selectively applying radio frequency (RF) energy. In such an embodiment, the initial dilation component incorporates at least one or more electrodes 21350 for electro-surgical cutting, which is electrically connected to an electro-surgical RF energy source outside the body of the patient. Alternatively, mechanical dilation components may be used instead of electro-surgical electrodes (e.g., a conical or progressively stepped dilator, Savary dilator, Maloney dilator, Hurst dilator, Soehendra dilator, Bougie-over-wire dilators).

The dilation component 21310 may incorporate at least one or more electrodes 21350 at or near its leading (distal) tip so that radio-frequency (RF) energy may be applied to the tissue to create a larger path through the tissue walls. Incorporating electrodes 21350 that are radially arrayed helps ensure that at least one electrode, of the more than one electrodes incorporated, contacts tissue and thus enlarges the puncture and enables the dilation component to advance into the target lumen. The initial dilation component may be positioned coaxially over the needle 21280, and slideable over the needle shaft, which acts as a guide. An advancement mechanism 21320 that positions the dilation component 21310 extends from the delivery system handle to the dilation component 21310 so that the user may manipulate and control the position of the dilation component 21310 during a procedure. Once the dilation component 21310 has been advanced over the needle shaft, through the wall of the access lumen (e.g., duodenum), and through the wall of the target lumen (e.g., gallbladder), the tract through the tissue walls accommodates the insertion of the inflatable shunt 2100.

In a configuration of the delivery system shown in FIG. 21, the inflatable shunt 2100, in its initial/deflated configuration, is positioned behind the leading edge of the dilation component 21310, and mounted in such a way that the advancement mechanism for positioning the dilation component 21310 also positions the inflatable shunt 2100. That is, advancing the dilation component 21310 into the target lumen (e.g., gallbladder) also advances at least a portion of the inflatable shunt 2100 (e.g., the distal anchor) into the target lumen (e.g., gallbladder). Inflation of the inflatable shunt's distal anchor may be performed with an inflation element 21330, which in the embodiment is a tube.

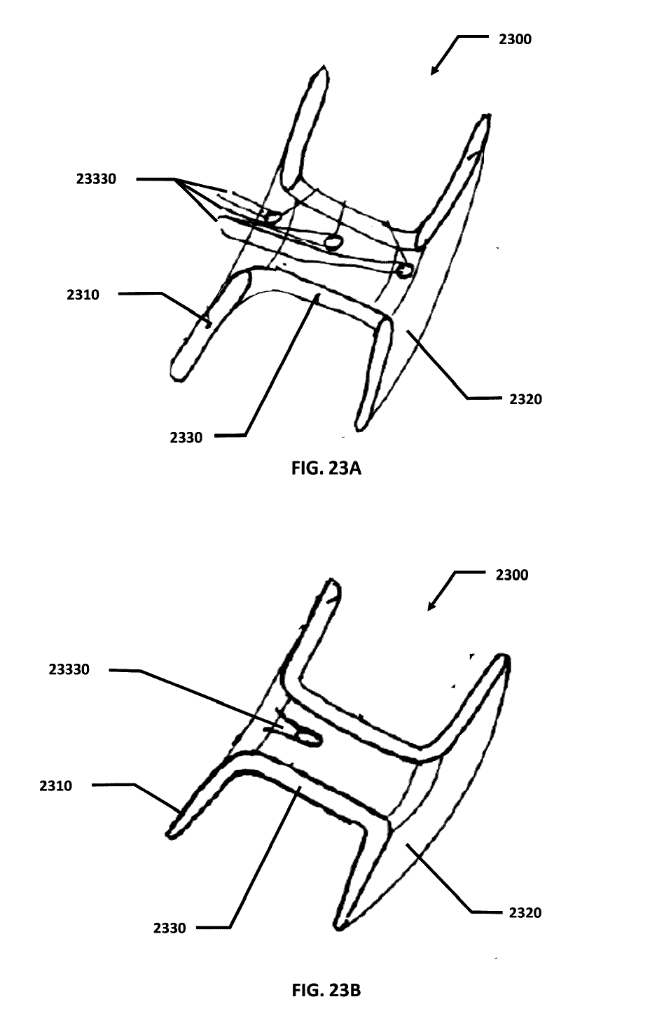

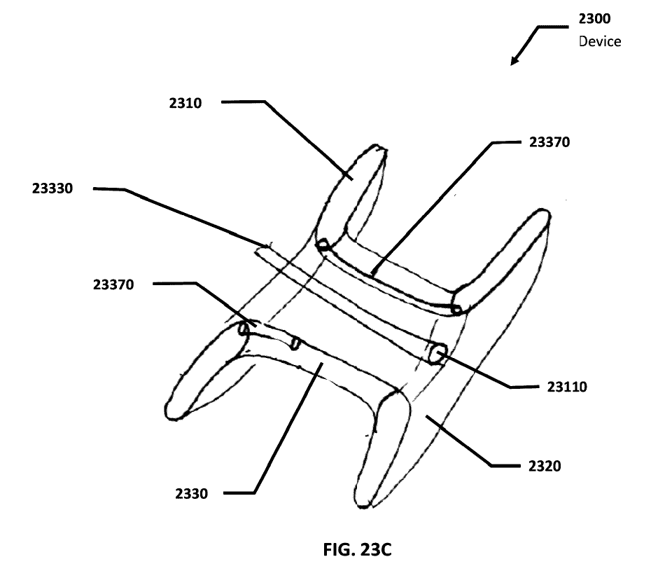

As shown in FIG. 23A, when the distal anchor 2320 is in position and inflated, tension may be applied to the shunt positioning component 21320, either individually or together with the needle 21280, so that the shunt's distal anchor 2320 is pulled against the inner surface of the lumen wall tissue (e.g., the inside surface of the gallbladder). With the distal anchor 2320 inflated, the risk of pulling the inflatable element 2300 back out through the dilated needle tract is reduced. When the proximal anchor 2310 of the inflatable shunt is in the desired position within the access lumen (e.g., the duodenum), the proximal anchor 2310 may be inflated with an inflation element 23330 (in an embodiment, a tube). Together, the distal anchor 2320 and proximal anchor 2310, when inflated, secure the inflatable element 2300 across the walls of the connected lumens, with the proximal anchor 2310 and the distal anchor 2320 positioned within one of each of the lumens. The central connecting section 2330 of the inflatable element 2300 may be inflated with an inflation element 23330 (in an embodiment, a tube).

In a variation of this method, the inflatable element 2300 may be inflated in a single step. In some cases, the inflatable element 2300 is configured with a single interior lumen for inflation FIG. 23B. In such cases, the initial position of the inflatable element 2300 is such that the distal anchor 2320 is inside the target lumen (e.g., gallbladder), the proximal anchor 2310 is inside the access lumen (e.g., duodenum), and the central connecting section 2330 extends across the tract in the walls of both the access and target lumens. Alternatively, an optional slideable sheath 22360 shown in FIG. 22) may be used to cover and constrain all or part of the inflatable element 2300, preventing individual portions from inflating, while other portions are uncovered and allowed to inflate. Further, in cases where all the inflatable sections of the inflatable shunt illustrated in FIG. 23 are connected and inflated together, the timing of the inflation of each section may be controlled and sequenced by connecting the sections with channels 23370 of varying size, inner cross-sectional area, and/or length adding a desired degree of resistance to the flow of the inflation material from the first chamber to a second chamber, and, if desired, a third chamber. In one example, if it is desired to inflate the distal anchor first 2320, followed by the proximal anchor 2310, or flange, and finally the central connecting section 2330, a single inflation port 23110 may be positioned to connect directly to the distal anchor 2320 with minimal flow restrictions. The distal anchor 2320 inflation chamber may be connected to the second inflatable anchor 2310 via a small channel 23370 (e.g., about 0.001″-0.010″), and this may in turn be connected to the inflation chamber of the central connecting section 2330 via another channel 23370 of similar, or dissimilar, size. In general, when a first chamber is inflated through the inflation port, it will first fill with inflation material at low pressure (insufficient to cause significant flow of inflation material to the second chamber). The introduction of additional inflation material into the first chamber will cause the pressure in the inflation chamber to increase to the point where the flow of inflation material into the second chamber occurs at a useful, selectable, rate (e.g., between about 1 second and 60 seconds). Once the second inflation chamber has filled with inflation material and pressure increases within the chamber to a desired amount, flow of inflation material into the third chamber increases to a desired amount, and the third chamber inflates at a desired rate (e.g., between about 1 second and 60 seconds). In this way, the process of sequentially inflating sections of an inflatable element 2300 that share a common inflation chamber, or more than one chamber connected via small channels, may be made to inflate sequentially in a controlled, timed, and selectable manner.

A small tract connecting the target and access lumens through the inner lumen of the inflatable shunt may be sufficient for the drainage of fluids, however larger tracts will provide improved drainage, and/or accommodate the insertion of items such as endoscopes or endoscopic tools into the target lumen (e.g., gallbladder), or for the extraction of material, such as gallstones, bile sludge, and the like. An inner diameter of the inner lumen of the inflatable element 2300 of at least 5 mm is desirable for enabling drainage and providing access for procedures through the inflatable element 2300, with still larger inner diameters of approximately 8-12 mm providing still better drainage and access, thereby enabling a greater number and variety of such treatments.

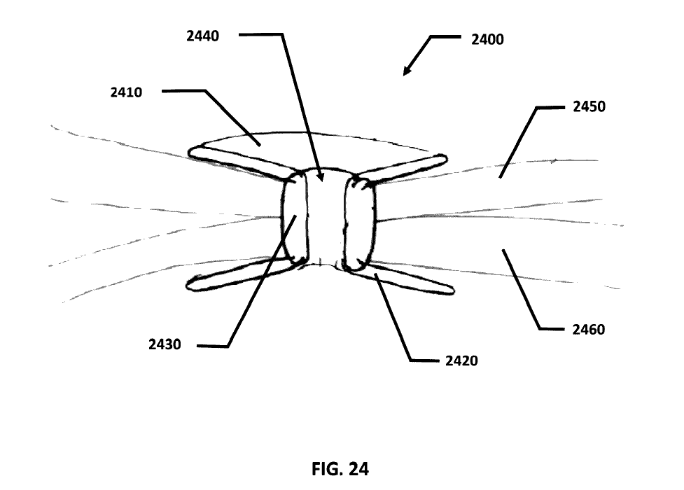

As depicted in FIG. 24, when the inflatable shunt 2400 having a first inflatable anchor 2410 and a second inflatable anchor 2420 and an aperture 2440 therethrough, is in position across the walls of the access and a first target lumen 2450 and a second target lumen 2460, if the connecting section 2430 of the inflatable shunt 2400 is configured to be inflatable, and further configured so that it forcefully expands when it is inflated, this may be sufficient to dilate the tract through the walls of the first target lumen 2450 and the second target lumen 2460 to the desired extent. Further, the forceful expansion of the connecting section 2430 may serve to seal the interface between the tissue of the first target lumen 2450 and the second target lumen 2460 and the connecting section 2430 of the inflatable shunt 2400, reducing the risk of leaks of fluid or other materials from the inside of a lumen to the outside (e.g., preventing the leakage of bile from the gallbladder into the peritoneum), or in the reverse direction. The connecting section 2430 can be centrally positioned. The characteristic expansion of the connecting section 2430 may be controlled so that the internal pressure delivered to the connecting section 2430 when inflated correlates to a known degree of expansion/dilation or dimensional size (e.g., inflating the center connecting section to a pressure of 5 atm may be selected to result in an outer diameter of 12 mm). Optionally, dilation may be achieved by a dilation balloon that is separate from the connecting section 2430 of the inflatable shunt 2400 but integral with it, so that it may be activated to dilate the tissue, and then deactivated, after which point the connecting section 2430 of the inflatable shunt 2400 holds the tract open and connects the access and target lumens.

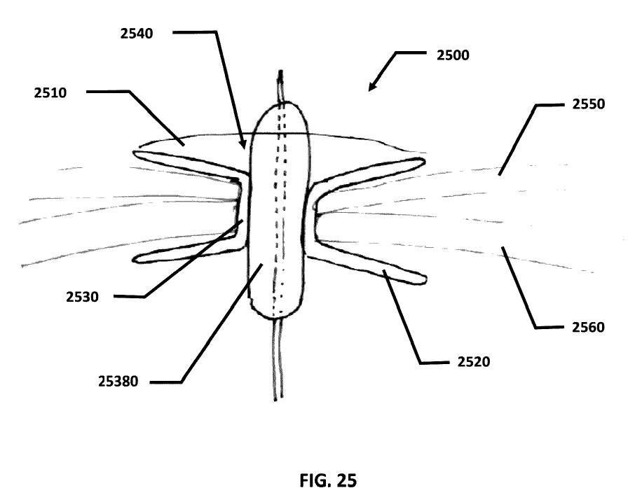

FIG. 25 shows a configuration where the connecting section 2530 is configured to expand radially outward when inflated, but not so forcefully as to dilate the tract through the tissue, which may serve to seal the interface between the tissue 2550, 2560 and the connecting section 2530 of the inflatable shunt device 2500 An additional component (e.g., a separate dilation balloon 25380) may be used to dilate the tract through the tissue once the inflatable shunt device 2500 is securely in place. For example, with the distal anchor 2520 and proximal anchor 2510 inflated in the desired locations, for delivery systems configured with at least a second dilation element (e.g., as shown in 21340), this is positioned within the inner lumen 2540 of the connecting section 2530 of the inflatable shunt device 2500 and activated to dilate the tissue surrounding the inflatable shunt device 2500. In an alternate embodiment, for a delivery system that does not incorporate a second dilation element, a separate dilation balloon (e.g., a Boston Scientific 10-11-12 CRE dilation balloon) may be inserted into the inner lumen 2540 of the inflatable shunt device 2500 and inflated to the desired pressure and related outer diameter (e.g., about 10-12 mm), to dilate the tissue 2550, 2560 surrounding the connecting section 2530. The dilation balloon 25380 may then be withdrawn from the inflatable shunt device 2500, and, in cases where the connecting section 2530 is inflatable, it may then be inflated. In other cases, the connecting section 2530 is not an inflatable component, and the procedure does not include a step for inflating it. This dilation step may be performed by other suitable dilation tools and methods, such as conical or progressively stepped dilators, Savary dilators, Maloney dilators, Hurst dilators, Soehendra dilators, Bougie-over-wire dilators).

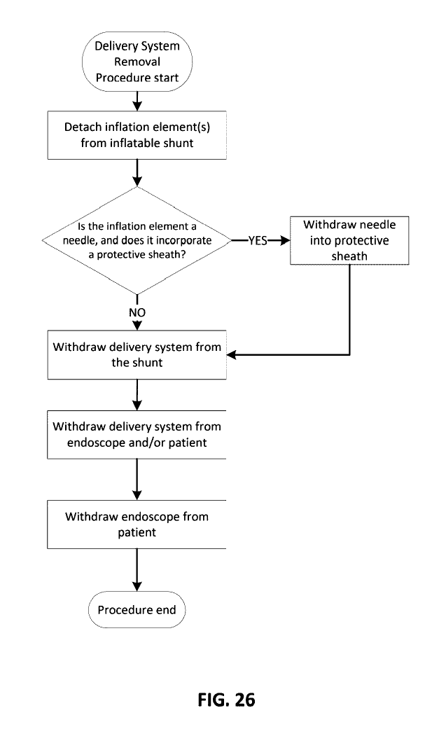

With the inflatable shunt in position and fully inflated, the delivery system may then be removed from the patient as shown in FIG. 26. The first step in the removal process is to detach the inflation element(s) from the inflated shunt. Depending on the configuration of the inflatable shunt and the mechanism(s) by which the shunt is inflated, this may involve, for example, detaching a tube or tubes through which fluid is caused to flow. In these cases, this may be achieved by applying tension to the inflation element, which pulls it free from the inflatable shunt. A number of inflation materials have been described elsewhere in this application, as well as the inflation elements used to deliver, manipulate, mix, and/or activate them, and it shall be understood that each of these embodiments will have its own mechanisms for sealing and detaching the inflation element from the inflated shunt. Once the inflation element is detached from the inflated shunt, the delivery system or separate tools comprising the delivery system, including the initial dilation component and the needle, may be withdrawn from the shunt. In an optional step, the needle is first withdrawn into a protective sheath, which may comprise at least a portion of the dilation component, so that the sharp tip of the needle does not injure tissue that it comes into contact with, nor damage the inflatable shunt. Once the initial dilation component and needle have been withdrawn from the inflated shunt, the delivery system may be withdrawn into the endoscope, and, in a final step, the endoscope may be withdrawn from the patient. At this point, the delivery procedure is complete.

It should be understood that the delivery method and delivery system are representative of only one approach for delivering the inflatable shunt, and that other devices and methods are possible and shall also be considered to be incorporated in this application. For example, rather than delivering the inflatable shunt over a needle, a guidewire may first be placed (for example, with a needle), and the guidewire may serve as the guide for a delivery system or a number of delivery tools that together comprise a delivery system. Alternatively, a guidance catheter may slide over the guidewire, and the guidance catheter may provide the access for a delivery system or a number of delivery tools that together comprise a delivery system. Alternatively, the inflatable shunt may be surgically implanted, rather than endoscopically, in which case, the surgeon delivers the shunt through an incision (which may be a minimally invasive incision, e.g., through a port or cannula) to a site for implantation. Though the delivery systems or tools and the implantation processes described throughout this application use the duodenum as the exemplar access lumen and the gallbladder as the exemplar target lumen, any two lumens that are initially in suitable proximity to each other (e.g., the stomach and a pancreatic pseudocyst, the stomach and the jejunum), and accessible by surgical, endoscopic, laparoscopic, or other suitable procedures, shall be considered as alternate and applicable examples of intended uses for the proposed devices.

Numerous methods and tools may be employed to remove the inflatable shunt from the implantation site when it is no longer needed or desired within the body. In cases where the shunt is inflated with material that is tolerated by the body (e.g., biocompatible materials, such as saline, CO₂), the balloon may be pierced, cut, punctured, ruptured, or otherwise compromised, so that the inflating material is released from the shunt. In some cases, the inflation port may be opened, unstopped, unglued, etc. Once the shunt has been deflated, it may be removed with an endoscopic tool, such as a grasper, a snare, a loop, to grasp the shunt and withdraw it from the body. Alternatively, the deflated shunt may be allowed to exit the body via a natural process, such as peristalsis. Further, if the shunt is bioabsorbable or resorbable, it may be left in place as long as desired, while it is dissolved, absorbed, or otherwise eliminated from the body.

In an alternate method for inflatable shunts that incorporate inflation/deflation ports to which inflation or deflation elements may be attached and detached, rather than compromising the integrity of the inflatable shunt to deflate it, a deflation element may be attached to one or more of the ports and the inflation material may be withdrawn from the shunt, e.g, via suction. The deflated shunt may then be removed from the body in any of the ways previously described.

In a further alternate method, inflatable shunts that are filled with activatable inflation materials may be deflated by deactivating the inflation material. For example, in cases where an activatable inflation material (e.g., one that includes nanotubes) is activated via illumination by light with specific properties (e.g., wavelength, intensity, duration, power, etc.) and deactivated via light with properties that may be the same or different from the light that activates the material, the inflation material may be withdrawn from the inflatable shunt once it is deactivated. The deflated shunt may then be removed from the body in any of the ways previously described.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An inflatable shunt comprising:
a first inflatable anchor at a first end having a first inflatable anchor interior surface and a first inflatable anchor exterior surface forming a first inflatable anchor lumen therebetween;
a second inflatable anchor at a second end having a second inflatable anchor interior surface and a second inflatable anchor exterior surface forming a second inflatable anchor lumen therebetween; and
an inflation port.

2. The inflatable shunt of claim 1 further comprising a connecting section positioned between the first inflatable anchor and the second inflatable anchor.

3. The inflatable shunt of claim 1 wherein the inflation port is positionable on at least one of the first inflatable anchor exterior, the second inflatable anchor exterior, and the connecting section.

4. The inflatable shunt of claim 1 wherein one or more inflation ports are positioned on a non-tissue contacting surface.

5. The inflatable shunt of claim 1 wherein the inflation port has a central lumen.

6. The inflatable shunt of claim 1 wherein the inflatable shunt has a central lumen.

7. The inflatable shunt of claim 2 wherein the connecting section is inflatable.

8. The inflatable shunt of claim 1 further comprising an activatable material within at least one of the first inflatable anchor lumen, and the second inflatable anchor lumen.

9. The inflatable shunt of claim 2 further comprising an activatable material within the connecting section.

10. The inflatable shunt of claim 1 wherein the inflatable shunt is inflated by one or more of a filling material and activation of an expandable material.

11. The inflatable shunt of claim 10 wherein at least one of the filling material and expandable material change hardness.

12. The inflatable shunt of claim 10 wherein the filling material is one or more of each of a compressible material and an incompressible material.

13. The inflatable shunt of claim 12 wherein the compressible material is one or more of air, inert gases, carbon dioxide, foam and gel.

14. The inflatable shunt of claim 12 wherein the incompressible material is one or more of liquid and gel.

15. The inflatable shunt of claim 1 wherein an exterior surface of the shunt has one or more of each of a surface texture, a ridge, a rib, an aperture, and a barb.

16. The inflatable shunt of claim 1 further comprising a plug.

17. The inflatable shunt of claim 1 wherein at least a portion of the shunt is biodegradable.

18. The inflatable shunt of claim 1 wherein a radial expansion of the inflatable shunt is selectable.

19. The inflatable shunt of claim 1 further comprising a valve positioned within a lumen of at least one of the inflatable shunt and the inflation port.

20. The inflatable shunt of claim 1 further comprising a structural component wherein the structural component either surrounds at least a portion of at least one of the first inflatable anchor, and the second inflatable anchor, or is incorporated into a portion of a wall of at least one of the first inflatable anchor, and the second inflatable anchor.

21. The inflatable shunt of claim 1 wherein the first inflatable anchor and the second inflatable anchor apply pressure to tissue positioned between a first inflatable anchor surface that is adjacent a second inflatable anchor surface.

22. A collapsible inflatable shunt comprising:
a first inflatable anchor at a first end having a first inflatable anchor interior surface and a first inflatable anchor exterior surface forming a first inflatable anchor lumen therebetween; and
a second inflatable anchor at a second end having a second inflatable anchor interior surface and a second inflatable anchor exterior surface forming a second inflatable anchor lumen therebetween,
wherein the inflatable shunt has a delivery configuration having a first profile and an deployed configuration having a second profile.

23.-43. (canceled)

44. An inflatable shunt comprising:
a first inflatable anchor at a first end having a first inflatable anchor interior surface and a first inflatable anchor exterior surface forming a first inflatable anchor lumen therebetween;
a second inflatable anchor at a second end having a second inflatable anchor interior surface and a second inflatable anchor exterior surface forming a second inflatable anchor lumen therebetween; and
a structural component wherein the structural component either surrounds at least a portion of at least one of the first inflatable anchor and the second inflatable anchor, or is incorporated into a portion of a wall of at least one of the first inflatable anchor and the second inflatable anchor.

45.-63. (canceled)

64. A method of treating biliary disease comprising:
creating a first otomy and a second otomy adjacent the first otomy;
delivering an inflatable shunt to the first otomy and the second otomy wherein the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, and an inflation port;
inflating the inflatable shunt;
anchoring the first inflatable anchor on a first tissue surface; and
anchoring the second inflatable anchor on a second tissue surface which does not contact the first tissue surface.

65.-70. (canceled)

71. A method of treating biliary disease comprising:
creating a first otomy and a second otomy adjacent the first otomy;
delivering an inflatable shunt to the first otomy and the second otomy wherein the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, wherein the inflatable shunt has a delivery configuration having a first profile and a deployed configuration having a second profile;
inflating the inflatable shunt;
anchoring the first inflatable anchor on a first tissue surface; and
anchoring the second inflatable anchor on a second tissue surface which does not contact the first tissue surface.

72.-77. (canceled)

78. A method of treating biliary disease comprising:
creating a first otomy and a second otomy adjacent the first otomy;
delivering an inflatable shunt to the first otomy and the second otomy comprising a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, and a structural component wherein the structural component either surrounds at least a portion of at least one of the first inflatable anchor, the second inflatable anchor and the connecting section, or is incorporated into a portion of a wall of at least one of the first inflatable anchor, the second inflatable anchor and the connecting section;
inflating the inflatable shunt;
anchoring the first inflatable anchor on a first tissue surface; and
anchoring the second inflatable anchor on a second tissue surface which does not contact the first tissue surface.

79-84. (canceled)

85. A method of removing an inflatable shunt comprising:
locating the inflatable shunt delivered to a first otomy and a second otomy adjacent the first otomy;
changing a profile of the inflatable shunt having a first inflatable anchor and a second inflatable anchor from a deployed profile to a removal profile wherein the deployed profile has a larger cross-section of at least one of the first inflatable anchor and the second inflatable anchor than a corresponding cross-section of the removal profile.

86.-87. (canceled)

88. A shunt inflation device comprising:
an inflation element wherein the inflation element engages an inflation port of an inflatable shunt at a distal end of the inflation element,
wherein the inflatable shunt is at least one of
the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, and an inflation port;
the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, and a shunt, wherein the inflatable shunt has a delivery configuration and a deployed configuration, and
the inflatable shunt comprises a first inflatable anchor at a first end having a first inflatable anchor interior surface and a first inflatable anchor exterior surface forming a first inflatable anchor lumen therebetween, a second inflatable anchor at a second end having a second inflatable anchor interior surface and a second inflatable anchor exterior surface forming a second inflatable anchor lumen therebetween, an inflation port and a structural component wherein the structural component either surrounds at least a portion of at least one of the first inflatable anchor, and the second inflatable anchor, or is incorporated into a portion of a wall of at least one of the first inflatable anchor, the second inflatable anchor and the connecting section.

89-99. (canceled)

100. A shunt inflation device comprising:
an inflation element wherein the inflation element engages an inflation port of an inflatable shunt at a distal end of an elongated tubular member connectable to the inflatable shunt wherein the inflation element is removable from the inflation port of the inflation port by at least one of sliding, twisting and severing.

101-107. (canceled)

108. A delivery system comprising:
a needle;
a dilation component;
an inflatable shunt;
a shunt delivery device.

109-110. (canceled)
